(19) United States
(12) Patent Application Publication
(10) Pub. No.: US 2021/0333279 A1
(43) Pub. Date: Oct. 28, 2021

(54) IDENTIFICATION AND TREATMENT OF TUMORS CHARACTERIZED BY AN OVEREXPRESSION OF THE NEONATAL FC RECEPTOR

(71) Applicants: Aarhus Universitet, Aarhus C (DK); Albumedix Ltd., Nottingham (GB)

(72) Inventors: Kenneth Alan Howard, Aarhus N (DK); Jason Cameron, Nottingham (GB); Maja Thim Larsen, Aarhus N (DK); Frederik Dagnæs-Hansen, Silkeborg (DK)

(21) Appl. No.: 16/346,491

(22) PCT Filed: Nov. 3, 2017
(86) PCT No.: PCT/DK2017/050363

§ 371 Date: Apr. 30, 2019

(30) Foreign Application Priority Data
Nov. 4, 2016 (DK) PA 2016 70870

(51) Int. Cl. G01N 33/574; G01N 33/566; A61P 35/00; A61K 38/38; A61K 51/08; A61K 49/00

(52) U.S. Cl. CPC G01N 33/574; G01N 33/566; A61K 49/0056; A61K 38/38; A61K 51/081; A61P 35/00

(57) ABSTRACT

In a first aspect, the invention relates to the identification of cancer types over-expressing the FcRn receptor. In a second aspect, the invention relates to the treatment of said cancer types. In further aspects, the invention relates to identification of subtypes of inflammatory diseases and treatments thereof. In an additional aspect, the invention relates to in vivo imaging of cancers over-expressing the FcRn receptor.

### TECHNICAL FIELD OF THE INVENTION

The present invention relates to the identification of tumours over-expressing the FcRn receptor. In particular, the present invention relates to treatments of such tumour subtypes. Further, the invention relates to (in vivo) imaging of tissue, such as tumours or inflammatory tissue, overexpressing the FcRn receptor.

### BACKGROUND OF THE INVENTION

Human serum albumin (HSA) is a natural carrier protein possessing multiple ligand binding sites with a plasma half-life ˜19 days, facilitated by interaction with the human neonatal Fc receptor (FcRn), that promotes it as a highly attractive drug delivery technology. HSA is naturally found in the blood plasma of mammals where it is the most abundant protein. It has important roles in maintaining the desired osmotic pressure of the blood and also in transport of various substances in the blood stream.

The neonatal Fc receptor (FcRn) “Brambell” is a bifunctional molecule that contributes to maintaining a high level of immunoglobulins of isotype G (IgGs) and albumin in serum in mammals. FcRn has been found to salvage albumin and IgG from intracellular degradation by a pH dependent mechanism, thus prolonging their serum half-lives. The plasma half-life of wild type human serum albumin (HSA) has been found to be approximately 19 days.

The use of albumin in drug delivery is well described. Therapeutic active agents may for example be conjugated to albumin (WO 2000/69902) or therapeutic active polypeptides may be fused genetically to albumin and expressed as chimeric proteins (WO 2001/79271 and WO 2003/59934) or small acidic or hydrophobic therapeutic active agents may associate reversibly to albumin (Kragh-Hansen et al, 2002, Biol. Pharm. Bull. 25, 695 and WO 2000/71079). Reversible binding to albumin can also be achieved for pharmaceutically beneficial compounds which have little or no albumin binding properties by associating such compounds to a moiety having albumin-binding properties (Kurtzhals et al, 1997, J. Pharm. Sci. 86: 1365, and WO 2010/065950). Kratz, 2008, J. Controlled Release 132, 171-183 provides a review of all these technologies. Benefits of using albumin for drug delivery are longer half-life and/or controlled release of a therapeutic agent and/or targeting to selective tissues or organs.

A number of natural albumin variants have been described. Otagiri et al, 2009, Biol. Pharm. Bull. 32(4), 527-534, discloses 77 known albumin variants. A number of other natural variants have been identified and some of these have been analyzed for FcRn binding (Andersen et at (2010), Clinical Biochemistry 43, 367-372; Galliano et al (1993) Biochim. Biophys. Acta 1225, 27-32; Minchiotti et al (1987) Biochim. Biophys. Acta 916, 41 1-418; Takahashi et al (1987) Proc. Natl. Acad. Sci. USA 84, 4413-4417; Carlson et al (1992). Proc. Nat. Acad. Sci. USA 89, 8225-8229; (Peach, R. J. and Brennan, S. O., (1991) Biochim Biophys Acta. 1097:49-54). The half-life of naturally occurring human albumin variants in a mouse model was described in Iwao, et al. (2007) B. B. A. Proteins and Proteomics 1774, 1582-1590. Furthermore, human made albumin variants with altered binding affinity to FcRn has been described in WO 2011/051489, WO 2011/124718, WO 2012/059486, WO 2012/150319, WO 2011/103076, WO 2012/112188 WO 2013/075066, WO 2014/072481 and WO2015/63611. WO 2013/135896 discloses albumin variants having one or more (e.g. several) alterations in Domain I and one or more (e.g. several) alterations in Domain III. WO 2015/036579 discloses albumin variants having one or more (e.g. several) alterations in Domain II. In sum, several albumin variants are known to the skilled person.

Cianga et al. (Human Immunology 64, 1152-1159 (2003)) discloses that breast cancer tumour cells express the FcRn receptor.

A current limitation in subtyping of cancers, is the identification of relevant cancer subtypes, which can be treated by treatment protocols optimized for the specific subtype. Hence, an improved method for subtyping cancers would be advantageous, and in particular, a more efficient and/or reliable treatment protocol of such cancer subtypes would be advantageous.

### SUMMARY OF THE INVENTION

FcRn expression in many different polarized epithelia in vitro systems that model the kidney, lung, placenta, and intestines has shown that FcRn expression endows upon the cell the ability to transcytose IgG bidirectionally. Whether FcRn undergoes recycling or transcytosis is still under active investigation for both IgG and albumin, however, it is the assertion of the inventors that over-expressed FcRn on diseased tissues can be targeted as a treatment regime.

As mentioned above, Cianga et al. discloses that breast cancer tumour cells express the FcRn receptor. However, Cianga et al. describes that the expression level of the FcRn receptor is “maintained” (thus not increased).

The present invention relates in one aspect to newly identified subtypes of cancers, which have up-regulated levels (over-expression) of the FcRn receptor. Such subtypes are considered an important discovery from a clinical point of view, since the presence of an up-regulated accessible (e.g. surface) receptor on cancer cells makes such subtypes promising targets for FcRn binding agents coupled to a therapeutic drug, diagnostic agent or imaging agent. Example 1 shows identification of such subtypes. Examples 2-4 show accumulation/targeting of engineered albumin variants in human xenografts after intravenous injection in mice.

An object of the present invention relates to the provision of methods for identification of cancer subtypes. In particular, it is an object of the present invention to provide a treatment protocol of the above-identified cancer subtypes.

A further aspect of the invention relates to a method of subtyping a cancer, staging a cancer, and/or predicting the risk of developing a cancer, the method comprising
providing a biological sample (for example, previously obtained) from a subject;
determining the level of FcRn in said sample; and
comparing said determined level to a reference level;
   wherein a higher level of FcRn in said sample compared to the reference level is indicative of an FcRn up-regulated subtype/stage; and wherein a level equal to or lower than said reference level is indicative of an FcRn normal subtype/stage or FcRn down-regulated subtype/stage;
   and/or
   wherein a higher level of FcRn in said sample compared to the reference level is indicative of an increased risk of developing a cancer; and wherein a level equal to or lower than said reference level is not indicative of an increased risk of developing a cancer.

The method may be carried out in vivo or in vitro, preferably in vitro.

In a preferred embodiment, the method is for subtyping a breast cancer or a colorectal cancer, the biological sample is a breast cancer sample or a colorectal cancer sample, and wherein a higher level of FcRn in said sample compared to the reference level is indicative of an FcRn up-regulated subtype/stage; and wherein a level equal to or lower than said reference level is indicative of an FcRn normal or FcRn down-regulated subtype. In another preferred embodiment, said method is for subtyping/identifying a cancer susceptible, or more susceptible, to treatment by an FcRn binding agent.

Another aspect of the present invention relates to a composition comprising an FcRn binding agent for use in the subtyping of a cancer, staging a cancer, and/or predicting the risk of developing a cancer,

wherein a higher level of FcRn in a biological sample compared to a reference level is indicative of an FcRn up-regulated subtype/stage; and wherein a level equal to or lower than said reference level is indicative of an FcRn normal subtype/stage;
and/or
wherein a higher level of FcRn in said sample compared to the reference level is indicative of an increased risk of developing a cancer; and wherein a level equal to or lower than said reference level is not indicative of an increased risk of developing a cancer. An FcRn level equal to or lower than said reference level may be indicative of a low, very low or substantially zero, risk of developing a cancer. Phrased in another way, a level equal to or lower than said reference level is indicative of a normal sample.

In a preferred embodiment, the composition is for subtyping a breast cancer or a colorectal cancer, the biological sample is abreast cancer sample or a colorectal cancer sample, and a higher level of FcRn in said sample compared to the reference level is indicative of an FcRn up-regulated subtype; and a level equal to or lower than said reference level is indicative of an FcRn normal or FcRn down-regulated subtype. In another preferred embodiment, said composition is for subtyping/identifying a cancer susceptible to treatment by an FcRn binding agent.

Yet another aspect of the present invention provides a composition comprising an FcRn binding agent coupled to a therapeutic agent, for use in the treatment of a cancer, wherein said cancer has up-regulated levels of FcRn compared to a reference level. Preferably, the cancer is a breast cancer or a colorectal cancer.

In yet a further aspect, the invention relates to a method of obtaining an image of a (FcRn positive) cancer (in vivo) in a subject, the method comprising the steps of:
a) delivering to a subject animal or human a pharmaceutically acceptable composition comprising of an FcRn binding agent coupled to a detectable moiety;
b) imaging the subject animal or human to identify a detectable signal from the FcRn binding agent coupled to a detectable moiety in the subject; and
c) generating an image of the detectable signal, thereby obtaining an image of a (FcRn positive) cancer in the subject animal or human.

### BRIEF DESCRIPTION OF THE FIGURES

FIG. 1 shows FcRn over-expression in cancer types compared to corresponding healthy bordering tissue. Large pictures show cancer tissue and inserts show healthy controls. A) Colon cancer. Scale bar corresponds to 250 um for both tumour tissue and normal bordering tissue (small insert) B) Breast cancer. Scale bar corresponds to 250 pm for tumour tissue and 100 pm for normal bordering tissue (small insert).

FIG. 2 shows FcRn expression in human cancer cell lines mouse xenografts. A, left) PXBC-3 pancreatic cancer cell; A, middle) HT 29 human colorectal adenocarcinoma cells; A, right) MCF-7 human breast cancer cells. B) FcRn expression investigated by qPCR. The obtained data was normalized to the pancreatic cancer PXBC-3.

FIG. 3. A) shows in vivo blood circulatory half-life of Alexa Fluor® 680-labelled albumin of FcRn low-binder (LB), wild-type (WT), or FcRn high-binder (HBI) in mice (N=3). Fluorescence intensity was measured with IVIS bioimager and data reported as mean fluorescence intensity (MFI) normalized to signal after 1 min. B) shows calculated half-life from 4-72 hours after injection

FIG. 4 shows fluorescence intensity (photons/sec/cm²/sr) ex vivo measurements for selected organs (Liver, Kidney, Spleen, Intestine, Heart, Lung, Skin, and Muscle) and Tumour of the treatment groups PBS (N=3), FcRn low-binder (LB) (N=3), wild-type (WT) (N=3), and FcRn high-binder (HBI) (N=3). For Region of Interest (ROI) the entire organ was chosen.

FIG. 5 shows fluorescence intensity (photons/sec/cm²/sr) ex vivo measurements for selected organs (Liver, Kidney, Spleen, Intestine, Heart, Lung, Skin, and Muscle) and Tumour of the treatment groups PBS (N=3), FcRn low-binder (LB), (N=3), wild-type (WT) (N=3), and FcRn high-binder (HBI) (N=3). For ROI, a constant area was chosen and applied to all organs and tumour.

FIG. 6 shows full body scans of mice of the different treatment groups PBS (N=3), FcRn low-binder (LB) (N=3), wild-type (WT) (N=3), FcRn high-binder (HBI) (N=3), after injecting luciferin-D on the day of termination of the study, 72 hours. Bioluminescence is depicted in the upper panel and fluorescence is depicted in the lower panel after spectral unmixing.

FIG. 7 shows fluorescence intensity per gram of tissue ((photons/sec/cm²/sr)/g) of tumours measured ex vivo in mice treated with PBS (N=3), FcRn low-binder (LB) (N=3), wild-type (WT) (N=3), and FcRn high-binder (HBI) (N=3). For ROI, the entire organ was selected.

FIG. 8 shows fluorescence per living cell in tumours using bioluminescence as a measure of bioluminescent luciferase-expressing tumour cells and dividing fluorescence intensity (photons/sec/cm²) by bioluminescence intensity (photons/sec/cm²). Tumours were measured ex vivo in from the following treatment groups PBS (N=3), FcRn low-binder (LB) (N=3), wild-type (WT) (N=3), and FcRn high-binder (HBI) (N=3). For ROI, the entire organ was selected.

FIG. 9. A) shows in vivo blood circulatory half-life of Alexa Fluor 680-labelled albumin of FcRn low-binder (LB, N=6), wild-type (WT, N=6), or FcRn high-binder II (HBII, N=7) in mice at time points 4 hours, 24 hours, 48 hours, and 72 hours. Fluorescence intensity measured with IVIS bio-imager and data reported as MFI normalized to signal after 1 min. Exponential regression curves are plotted. B) shows in vivo blood circulatory half-life of Alexa Fluor 680-labelled albumin of FcRn low-binder (LB, N=6), wild-type (WT, N=6), or FcRn high-binder II (HBII, N=7) in mice in the initial phase up to 24 hours. Exponential regression curves are plotted for each albumin variant. Data is reported as MFI normalized to signal after 1 min. C) shows in vivo blood circulatory half-life of Alexa Fluor 680-labelled albumin of FcRn low-binder (LB, N=6), wild-type (WT, N=6), or FcRn high-binder II (HBII. N=7) in mice at time points from 24-72 hours. Exponential regression curves are plotted for each albumin variant. Data is reported as MFI normalized to signal after 1 min. D) Half-life values for Alexa Fluor 680-labelled albumin variants FcRn low-binder (LB), wild-type (WT), and FcRn high binder II (HBII) calculated from exponential curve fits. Half-life is given in hours and R² values are given from curve fit.

FIG. 10 shows biodistribution of PBS and albumin variants FcRn low-binder (LB, N=6), wild-type (WT) (N=6), and FcRn high binder II (HBII) (N=7) in organs and tumours. Fluorescence after spectral unmixing is given as average radiance (photons/sec/cm²/sr) of a Region of Interest of constant size.

FIG. 11 shows accumulation of Alexa Fluor 680-labelled albumin variants FcRn low-binder (LB) (N=6), wild-type (WT) (N=6), and FcRn high binder II (HBII) (N=7) in tumour measured ex vivo of tumour only and data is reported as MFI normalized to signal from tumour of PBS treated mice (N=3) and with a region of interest of constant size. *p<0.05, calculated by unpaired t-test.

FIG. 12 shows fluorescence intensity per gram of tissue ((photons/sec/cm²/sr)/g) of tumours measured alone ex vivo in mice treated with PBS (N=3), FcRn low-binder (LB) (N=6), wild-type (WT) (N=6), and FcRn high-binder II (HBII) (N=7). An ROI of constant size was applied to all tumours and data is reported as MFI normalized to signal from tumour signal of PBS treated mice (N=3).

FIG. 13 shows fluorescence intensity per gram of tissue ((photons/sec/cm²/sr)/g) of tumours measured alone ex vivo in mice treated with PBS (N=3), FcRn low-binder (LB) (N=6), wild-type (WT) (N=6), and FcRn high-binder II (HBII) (N=7). An ROI of entire tumour was applied to all tumours and data is reported as MFI normalized to signal from tumour signal of PBS treated mice (N=3).

FIG. 14. A) shows 21 hours scan of mice treated with PBS (N=3), FcRn low-binder (LB) (N=6), wild-type (WT) (N=6), or FcRn high-binder II (HBII) (N=7). Spectral unmixing was not performed at this time point as autofluorescence was very low. The pictures are from excitation wavelength at 675 nm and emission wavelength 720 nm. B) shows 72 hours scan of mice treated with PBS (N=3), FcRn low-binder (LB) (N=6), wild-type (WT) (N=6), or FcRn high-binder II (HBII) (N=7). Spectral unmixing was performed.

FIG. 15 shows representative pictures of the FcRn expression in four rheumatoid arthritis samples. The scale bar for A), B), and C) is 250 pm and for D) 100 pm.

FIG. 16: FcRn expression in different cancer tissues and corresponding normal tissue. Biopsies are from the tumour site in cancer patients. Sections are stained with antibodies against hFcRn and scored from negative to high expression by an experienced pathologist. Patient number (n) differs for each cancer type. A) Colorectal cancer, B) Breast cancer subtype Luminal B, Breast cancer subtype Triple Negative, D) Kidney cancer, E) Pancreatic cancer, F) Cervical cancer, G) Head and neck cancer, H) Lung cancer, I) Ovarian cancer, J) Bladder cancer.

FIG. 17 shows full body scans of mice of the different treatment groups PBS, wild-type (WT), FcRn high-binder (HBI), after injecting luciferin-D on the day of termination of the study. 72 hours. AlexaFluor680-labelled Albumin fluorescence is shown in the upper panel, and cellular bioluminescence is shown in the lower panel after spectral unmixing. Images are selected images also presented in FIG. 6.

FIG. 18: AlexaFlour488 labelled albumin variants uptake in human FcRn-expressing HT-29 (black bars) and HT-29 human FcRn knockout (white bars) cells. Mean Fluorescence intensity detected by flow cytometry after exposure to AlexaFluor488-labelled recombinant albumin variants for 2 hours in HBSS; FcRn low binder (LB), wild-type (WT), FcRn high binder I (HBI), and FcRn high binder II (HBII). Data is normalized to non-treated cells (NT). Error bars are depicted as standard deviation. Insert shows FcRn expression western blot analysis for HT-29 WT and HT-29 FcRn knockout. FcRn band is detected at ˜40 kDa depicted by arrow.

FIG. 19: Western blot of MDAMB231/Luc cell line and HT-29 cell line. FcRn expression is detected at ˜40 kDa depicted by arrow.

The present invention will now be described in more detail in the following.

### DETAILED DESCRIPTION OF THE INVENTION

### Definitions

Prior to discussing the present invention in further detail, the following terms and conventions will first be defined:

Colorectal Cancer

Colorectal cancer (CRC), also known as bowel cancer, or colon cancer, is the development of cancer from the colon or rectum (parts of the large intestine). It is due to the abnormal growth of cells that have the ability to invade or spread to other parts of the body.

Breast Cancer

Breast cancer is cancer that develops from breast tissue. Signs of breast cancer may include a lump in the breast, a change in breast shape, dimpling of the skin, fluid coming from the nipple, or a red scaly patch of skin. Examples of breast cancer subtypes are subtype Luminal B and subtype Triple Negative. Luminal B breast cancer is hormone-receptor positive (estrogen-receptor and/or progesterone-receptor positive), and either HER2 positive or HER2 negative with high levels of Ki-67. Luminal B cancers generally grow slightly faster than luminal A cancers and their prognosis is slightly worse. Triple-negative/basal-like breast cancer is hormone-receptor negative (estrogen-receptor and progesterone-receptor negative) and HER2 negative. This type of cancer is more common in women with BRCA1 gene mutations.

Albumin

The term ‘albumin’ means a protein having the same and/or very similar three-dimensional (tertiary) structure as human serum albumin (‘HSA’, SEQ ID NO: 1) or one or more HSA domain and has similar properties to HSA or to the relevant domain or domains. Similar three-dimensional structures are, for example, the structures of HSA. Some of the major properties of albumin are i) its ability to regulate plasma volume (oncotic activity), ii) a long plasma half-life of around 19 days ±5 days, iii) binding to FcRn, iv) ligand-binding, e.g. binding of endogenous molecules such as acidic, lipophilic compounds including bilirubin, fatty acids, hemin and thyroxine v) binding of small organic compounds with acidic or electronegative features e.g. drugs such as warfarin, diazepam, ibuprofen and paclitaxel. Not all of these properties need to be fulfilled in order to characterize a protein or fragment as an albumin. If a fragment, for example, does not comprise a domain responsible for binding of certain ligands or organic compounds the variant of such a fragment will not be expected to have these properties either. Preferably, albumin has at least 60% sequence identity to SEQ ID NO: 1, for example at least 65, 70, 75, 80, 85, 90, 91, 92, 93, 94, 95, 96, 97, 98, 98.2, 98.4, 98.6, 98.8, 99, 99.2, 99.4, 99.6 or 99.8% identity to SEQ ID NO: 1. Sequence identity may be calculated according to WO 2015/036579, particularly by using the Needleman-Wunsch algorithm as described on page 11.

HSA Variant

The term “HSA variant” or “variant HSA” means a polypeptide derived from a human serum albumin comprising an alteration, i.e., a substitution, insertion, and/or deletion, at one or more (several) positions. A substitution means a replacement of an amino acid occupying a position with a different amino acid; a deletion means removal of an amino acid occupying a position; and an insertion means adding 1-3 amino acids adjacent to an amino acid occupying a position. The variant may also be a functional fragment of HSA. Fragments may consist of one uninterrupted sequence derived from albumin or may comprise two or more sequences derived from different parts of the albumin. The fragments according to the invention may have a size of more than approximately 100 amino acid residues, preferably more than 150 amino acid residues, more preferred more than 200 amino acid residues, more preferred more than 300 amino acid residues, even more preferred more than 400 amino acid residues and most preferred more than 500 amino acid residues.

Relative to WT HSA, the variant may have a higher binding affinity to FcRn or a weaker binding affinity FcRn. Preferably the FcRn is human FcRn (hFcRn), more preferably soluble human FcRn (shFcRn). Examples of albumin variants useful to this invention are:
albumins having at least 70% identity to HSA (SEQ ID NO: 1) and having a mutation at a position corresponding to K573 of SEQ ID NO: 1 e.g. HSA-K573P (High-binder I) (HBI) (SEQ ID NO: 4)
albumins having at least 70% identity to HSA (SEQ ID NO: 1) and having a mutation at position corresponding to E492, K573, K574, and Q580 of SEQ ID NO: 1 e.g. HSA-E492G, K573P, K574H, Q580K (High-binder II) (HBII) (SEQ ID NO: 5)
albumins having at least 70% identity to HSA (SEQ ID NO: 1) and having a mutation at a position corresponding to K500 of SEQ ID NO: 1 e.g. HSA-K500A (Low-binder) (LB) (SEQ ID NO: 6)

In addition, a null-binder may in some cases be useful. An example is:
albumins having at least 70% identity to HSA (SEQ ID NO: 1) and having a mutation at a position corresponding to K500 and H464 of SEQ ID NO: 1 e.g. HSA-K500A, H464Q (Null-binder) (SEQ ID NO: 7)

Examples of albumin variants used in the example section are:
HSA-K573P (High-binder I) (HBI) (SEQ ID NO: 4)
HSA-E492G, K573P, K574H, Q580K (High-binder II) (HBII) (SEQ ID NO: 5)
HSA-K500A (Low-binder) (LB) (SEQ ID NO: 6)

In addition, a null-binder may in some cases be relevant. An example is:
HSA-K500A, H464Q (Null-binder) (SEQ ID NO: 7)

Antibody

The term ‘antibody’ or ‘antibody molecule’ includes whole antibodies (e.g. Immunoglobulin G (IgG), Immunoglobulin A (IgA), Immunogolbulin E (IgE), Immunoglobulin M (IgM), or Immunoglobulin D (IgD)), and antibody fragments such as Fab, F(ab′)2, Fab3, scFv, Fv, dsFv, ds-scFv, Fd, dAbs, TandAbs, minibodies, diabodies, tribodies, tetrabodies, vH domain, vL domain, vHH domain, Nanobodies, Affibodies, IgNAR variable single domain (v-NAR domain), fragments thereof, and multimers thereof and bispecific antibody fragments. Antibodies include monoclonal antibodies (‘mAbs’), polyclonal antibodies, and chimeric antibodies.

FcRn

The neonatal Fc receptor (FcRn), also known as the Brambell receptor, is a protein that in humans is encoded by the FCGRT gene. The human neonatal Fc receptor comprises an Fc receptor (SEQ ID NO: 2) associated with beta-2-microglobulin (SEQ ID NO: 3). The Fc receptor is similar in structure to the MHC class I molecule. Thus, the FcRn detected, targeted and/or imaged is preferably a human FcRn.

Mammal

The term “mammal” includes humans, domestic and farm animals (e.g. cows, sheep, pigs, horses), and zoo, sports (e.g. dogs or horses), or pet animals (e.g. dogs, cats, rabbits). Preferably, the mammal is human. The biological samples according to the invention are preferably provided from a human.

Reference Level

In the context of the present invention, the term “reference” relates to a standard in relation to quantity, quality or type, against which other values or characteristics can be compared, such as a standard curve.

In the present invention, the reference values may be the expression levels of FcRn. A set of reference data may be established by collecting the reference values for a number of samples. As will be obvious to those of skill in the art, the set of reference data will improve by including increasing numbers of reference values.

In one preferred embodiment of the present invention, the reference means is an internal reference means and/or an external reference means. In the present context the term “internal reference means” relates to a reference which is not handled by the user directly for each determination, but which is incorporated into a device for the determination of the concentration/level of FcRn, whereby only the ‘final result’ or the ‘final measurement’ is presented. The terms the “final result” or the “final measurement” relate to the result presented to the user when the reference value has been taken into account. In the present context, the term “external reference means” relates to a reference which is handled directly by the user in order to determine the concentration/level of FcRn, before obtaining the “final result”. In yet a further embodiment of the present invention external reference means are selected from the group consisting of a table, a diagram and similar reference means where the user can compare the measured signal to the selected reference means. To determine whether the subject has an FcRn over-expressing subtype, a cut-off must be established. This cut-off may be established by the laboratory, the physician or on a case-by-case basis for each subject. The cut-off level could be established using a number of methods, including: percentiles, mean plus or minus standard deviation(s); multiples of median value; patient specific risk or other methods known to those skilled in the art.

The multivariate discriminant analysis and other risk assessments can be performed on the commercially available computer program statistical package Statistical Analysis System (manufactured and sold by SAS Institute Inc.) or by other methods of multivariate statistical analysis or other statistical software packages or screening software known to those skilled in the art.

As obvious to one skilled in the art, in any of the embodiments discussed above, changing the cut-off level could change the results of the discriminant analysis for each patient.

When expression levels of FcRn are compared to a reference level, they can either be different (above or below the reference value) or equal. However, using today's detection techniques an exact definition of different or equal result can be difficult because of noise and variations in obtained expression levels from different samples. Hence, the usual method for evaluating whether two or more expression levels are different or equal involves statistical analysis.

Statistical analysis enables evaluation of significantly different expression levels and significantly equal expressions levels. Statistical methods involve applying a function/statistical algorithm to a set of data. Statistical theory defines a statistic as a function of a sample where the function itself is independent of the sample's distribution: the term is used both for the function and for the value of the function on a given sample. Commonly used statistical tests or methods applied to a data set include t-test, f-test or even more advanced tests and methods of comparing data. Using such tests or methods enables a conclusion of whether two or more samples are significantly different or significantly equal.

The significance may be determined by the standard statistical methodology known by the person skilled in the art. The chosen reference level may be changed depending on the mammal for which the test is applied. The chosen reference level may be changed if desiring a different specificity or sensitivity as known in the art.

As used herein the sensitivity refers to the measures of the proportion of actual positives, which are correctly identified as such—in analogy with a diagnostic test, i.e. the percentage of mammals or people overexpressing FcRn. Usually the sensitivity of a test can be described as the proportion of true positives of the total number. As used herein the specificity refers to measures of the proportion of negatives, which are correctly identified—i.e. the percentage of mammal with an FcRn level equal to or below normal. The ideal diagnostic test is a test that has 100% specificity, i.e. only detects mammals which over-express FcRn and, therefore, no false positive results, and has 100% sensitivity

For any test, there is usually a trade-off between each measure. For example in a manufacturing setting in which one is testing for faults, one may be willing to risk discarding functioning components (low specificity), in order to increase the chance of identifying nearly all faulty components (high sensitivity). This trade-off can be represented graphically using a receiver operating characteristic (ROC curve).

Thus, in an embodiment the subtypes according to the present invention has an up-regulated level (over-expression) of FcRn of at least 2×, such as at least 4×, such as at least 6×, such as at least 10× compared to reference tissue. The preferred method for determining FcRn expression is the method used in the example section.

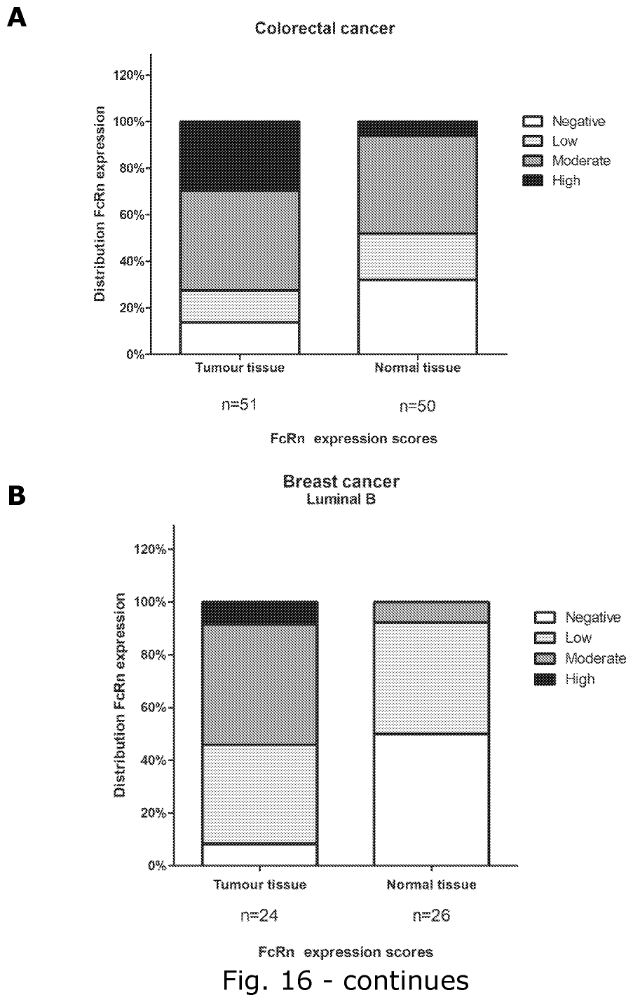

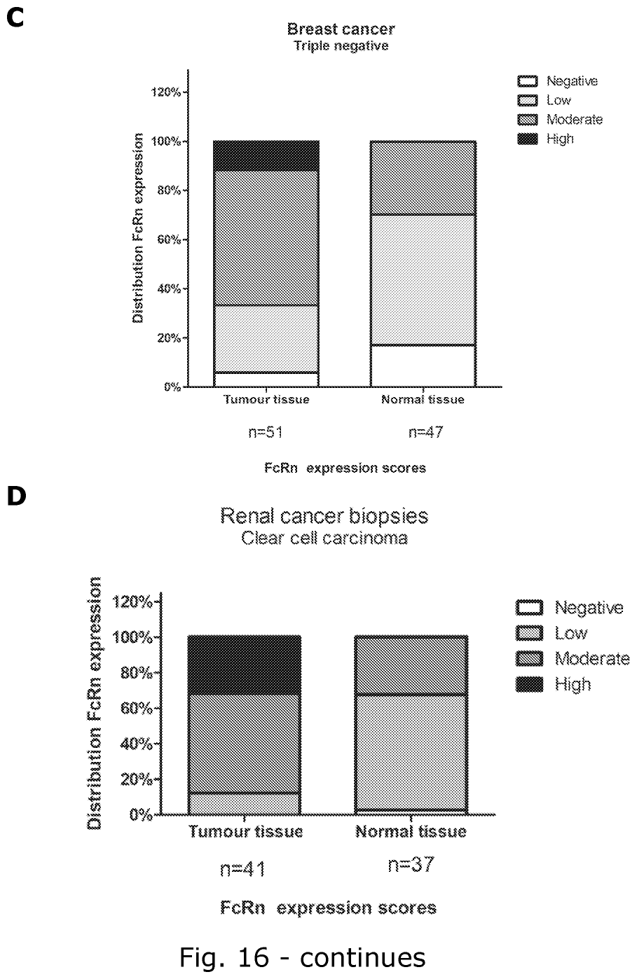

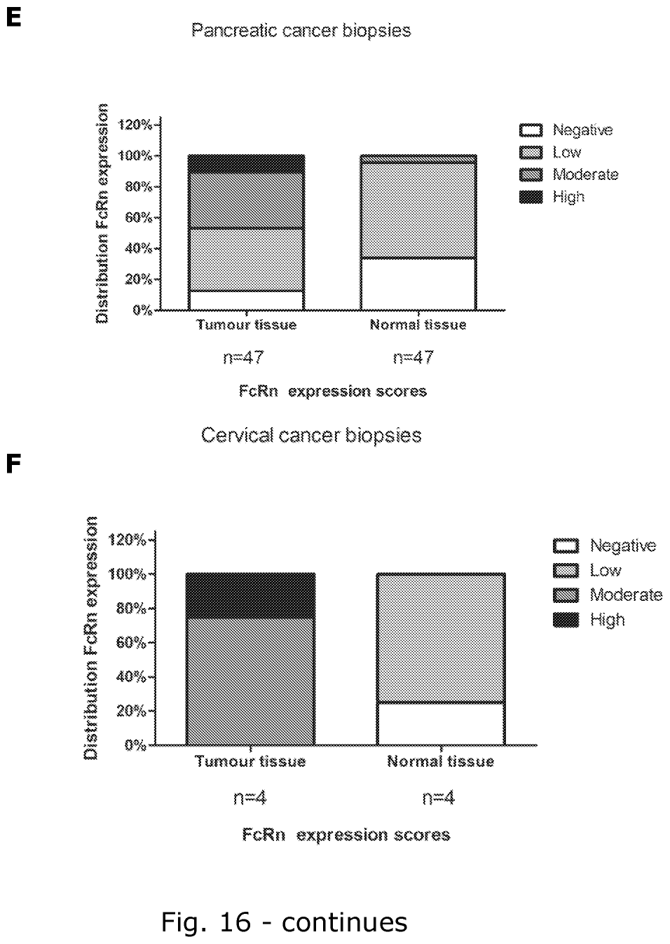

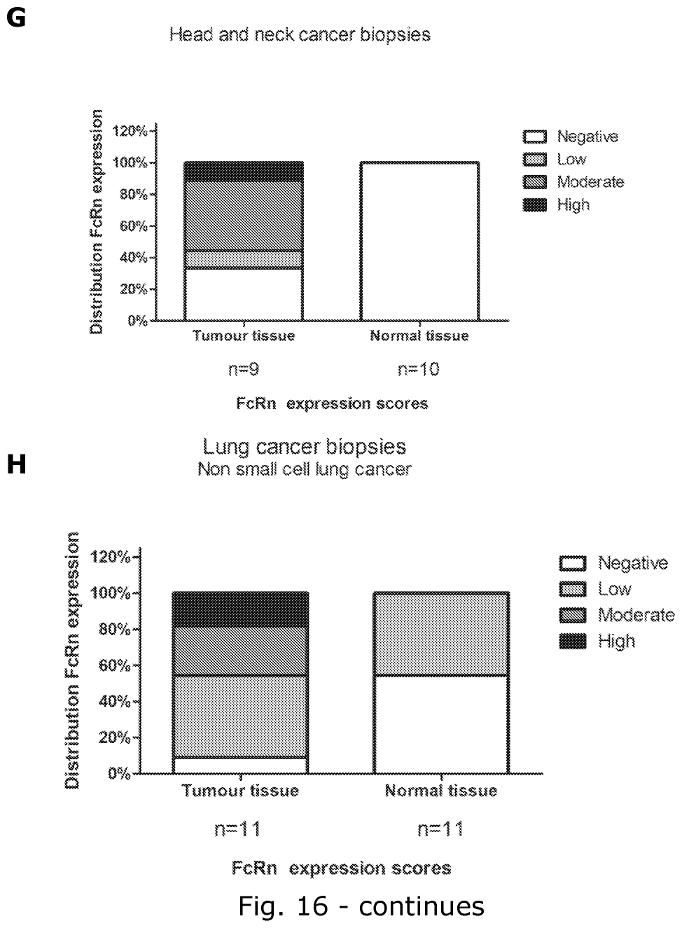

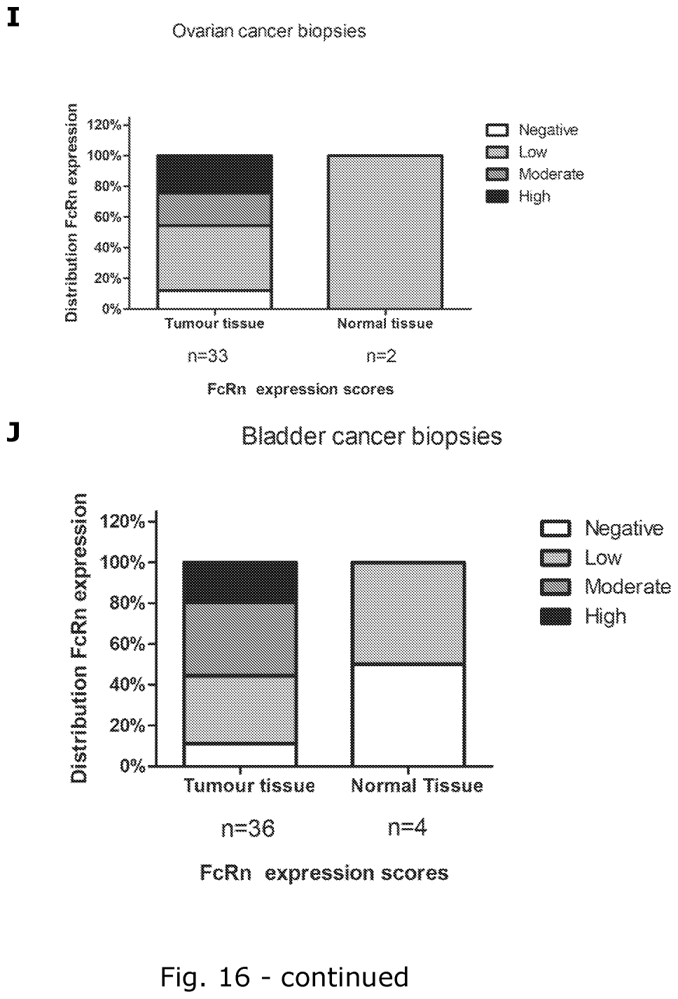

As seen from the example section the “cut-off value” can also depend on how different levels of expression are grouped. For example in FIG. 16, expression levels of FcRn are divided into negative, low, moderate or high. However, different groupings could be selected by the skilled person.

Binding Affinity

The term “binding affinity” generally refers to the strength of the sum total of the non-covalent interactions between a single binding site of a molecule (e.g., IgG or albumin) and its binding partner (e.g., an antigen or FcRn). Unless indicated otherwise, as used herein, “binding affinity” refers to intrinsic binding affinity, which reflects a 1:1 interaction between members of a binding pair (e.g., albumin and FcRn). The affinity of a molecule (X) for its partner (Y) can generally be represented by the equilibrium dissociation constant (KD), which is calculated as the ratio k_{off}/kₒₙ (kd/ka). Binding affinity can be measured by methods known in the art. A preferred method is surface plasmon resonance (SPR) for example using a Biacore (GE Healthcare) instrument as exemplified herein. The binding affinity of endogenous pairs of FcRn and albumin (e.g. HSA to hFcRn, dog albumin to dog FcRn and so forth) generally ranges from 0.2 to 3.2 micro Molar.

Binding affinity may (as an example) be expressed as an albumin or conjugate/fusion/association thereof having a KD to FcRn (e.g. shFcRn) that is lower than the corresponding KD for HSA (higher, i.e. stronger binding). Thus, in an embodiment, the KD for the “FcRn binding agent” according to the invention (such as an albumin, an albumin variant or conjugate/fusion/association thereof) is less than 0.9×KD for HSA to FcRn, more preferred less than 0.5×KD for HSA to FcRn, more preferred less than 0.1×KD for HSA to FcRn, even more preferred less than 0.05×KD for HSA to FcRn, even more preferred less than 0.02×KD for HSA to FcRn, even more preferred less than 0.01×KD for HSA to FcRn and most preferred less than 0.001×KD for HSA to FcRn (where X means ‘multiplied by’). Preferably the “FcRn binding agent” is an albumin or albumin variant according to the invention.

Alternatively, the KD to FcRn may be higher than the corresponding KD for HSA to FcRn (lower binding). Thus, in an embodiment, the KD for the the “FcRn binding agent” is more than 2×KD for HSA to FcRn, more preferred more than 5×KD for HSA to FcRn, more preferred more than 10×KD for HSA to FcRn, even more preferred more than 25×KD for HSA to FcRn, most preferred more than 50×KD for HSA to FcRn. It is noted the preferably the “FcRn binding agent” is an albumin or albumin variant according to the invention.

In most instances, it is preferred that a variant with a higher binding affinity to FcRn is used in the aspects of the present invention.

When determining and/or comparing KD, one or more (e.g. several) (and preferably all) of the following parameters may be used:
Instrument: Biacore 3000 instrument (GE Healthcare)
Flow cell: CM5 sensor chip
FcRn: human FcRn, preferably soluble human FcRn, optionally coupled to a tag such as Glutathione S Transferase (GST) or Histidine (His), most preferably His such as 6 histidine residues at the C-terminus of the beta-2-microglobulin.
Quantity of FcRn: 1200-2500 RU
Coupling chemistry: amine coupling chemistry (e.g. as described in the protocol provided by the manufacturer of the instrument).
Coupling method: The coupling may be performed by injecting 20 μg/ml of the protein in 10 mM sodium acetate pH 5.0 (GE Healthcare). Phosphate buffer (67 mM phosphate buffer, 0.15 M NaCl, 0.005% Tween 20) at pH 5.5) may be used as running buffer and dilution buffer. Regeneration of the surfaces may be done using injections of HBS-EP buffer (0.01 M HEPES, 0.15 M NaCl, 3 mM EDTA, 0.005% surfactant P20) at pH 7.4 (Biacore AB).
Quantity of injection of test molecule (e.g. HSA or variant) 20-0.032 μM
Flow rate of injection: constant, e.g. 30 μl/ml
Temperature of injection: 25° C.
Data evaluation software: BIAevaluation 4.1 software (BIAcore AB).

The “pH dependence ratio” a measure of pH dependency was assessed as a ratio of response at equilibrium at pH 7.4 over pH 5.5×100.

Modified or Modification

The term “modified” or “modification” in relation to albumin means to change the albumin by adding or deleting molecules unrelated to the amino acid sequence of the albumin, e.g. removing fatty acids or adding a partner molecule. The albumin can in in particular be modified by conjugation, fusion or association of a partner. Changes to the amino acid sequence of the albumin (e.g. SEQ ID NO: 1) is termed “variants” and are not considered modifications.

Conjugation

The term “conjugated”, “conjugate”, or “conjugation” below exemplified in relation to albumin refers to WT HSA or a variant HSA or a fragment thereof, which is conjugated to a conjugation partner such as a beneficial agent, e.g. a therapeutic agent and/or diagnostic agent. Conjugation can be made to the N-terminal and/or C-terminal of the albumin, but can alternatively or in addition be made to one or more (several) suitable amino acid positions within the albumin. In particular, cysteine residues which are not involved in disulfide bonds are suitable for conjugation. WO 2009/126920, WO 2010/059315 and WO 2010/092135 (hereby incorporated by reference) describe variant albumins with additional cysteine residues suitable for conjugation. Techniques for conjugating a conjugation partner to an albumin or fragment thereof are known in the art. WO 2009/019314 discloses examples of techniques suitable for conjugating a conjugation partner, e.g. a therapeutic agent, to a polypeptide which techniques can also be applied to the present invention. Furthermore, page 37 to 44 of WO 2009/019314 (hereby incorporated by reference) discloses examples of compounds and moieties that may be conjugated to transferrin and these compounds and moieties may also be conjugated to an albumin variant of the present invention. It is to be understood that the above is also the case for other FcRn binding partners different from albumins according to the present invention.

Fused

The term “fused” or “fusion” described below in relation to albumin refers to WT HSA or a variant HSA or a fragment thereof which is genetically fused to a fusion partner such as a beneficial agent e.g. a therapeutic polypeptide and/or diagnostic polypeptide. Fusions are normally made at either the N-terminal or C-terminal of the albumin, or sometimes at both ends. Fusions can in principle, alternatively or in addition, be made within the albumin molecule, in that case it is preferred to locate the fusion partner between domains of albumin. For example, a fusion partner may be located between Domain I and Domain II and/or between Domain II and Domain III. Teachings relating to fusions of albumin or a fragment thereof are known in the art and the skilled person will appreciate that such teachings can also be applied to the present invention. Table 1 of WO 2001/79271, Table 1 (page 1 1) of WO 2001/79258, Table 1 (page 11) of WO 2001/79442, Table 1 (page 12) of WO 2001/79443, Table 1 (page 11) of WO 2001/79443, Table 1 of WO 2003/060071, Table 1 of WO 2003/59934, Table 1 of WO 2005/003296, Table 1 of WO 2007/021494 and Table 1 of WO 2009/058322 (all tables are hereby incorporated by reference) contain examples of fusion partners, e.g. therapeutic polypeptides, that may be fused to albumin or fragments thereof, and these examples apply also to the present invention. It is to be understood that the above is also the case for other FcRn binding partners different from albumins according to the present invention.

Association

The term “associated”, “associate”, or “association” exemplified in this section in relation to albumin refers to a composition comprising WT HSA or variant HSA or a fragment thereof and an association partner, such as a therapeutic agent and/or diagnostic agent, bound or associated to the albumin or fragment thereof by non-covalent binding. An example of such an associate is an albumin and a lipid associated to the albumin by a hydrophobic interaction. Such associates are known in the art and they may be prepared using well known techniques. Molecules which are suitable for association with albumin are known in the art, preferably they are acidic, lipophilic and/or have electronegative features. Examples of such molecules are given in Table 1 of Kragh-Hansen et al, 2002, Biol. Pharm. Bull. 25, 695 (hereby incorporated by reference). Furthermore, WO 2000/071079 describes the association of albumin with paclitaxel and paclitaxel is included in the present invention.

The association partner may also be associated to the therapeutic agent and/or diagnostic agent through micro- or nanoparticles wherein the therapeutic agent and/or diagnostic agent is attached to or incorporated in the particle. It is to be understood that the above is also the case for other FcRn binding partners different from albumins according to the present invention.

Wild-Type (WT)

The term “wild-type” (WT) in relation to e.g. albumin or FcRn means an albumin or FcRn having the same amino acid sequence as the albumin or FcRn naturally found in an animal or in a human (the endogenous gene sequence of the animal or human). It is understood that WT albumin or WT FcRn is without genetic alterations produced by human intervention for example by gene knock-out/knock-in as in the production of transgenic animals. SEQ ID NO: 1 is a mature WT albumin from Homo sapiens.

Therapeutic Agent

The term “therapeutic agent”, “therapeutic compound”, “therapeutic molecule” or “drug” is used interchangeably and refers to a chemical compound, a mixture of chemical compounds, or a biological macromolecule (e.g. a peptide, protein, lipid, nucleic acid (e.g. DNA or RNA), virus) or a biological macromolecule in association with a chemical compound. Therapeutic agents include agents that can either prevent, improve or cure a medical condition. The therapeutic agent may be purified, substantially purified or partially purified. An “agent”, according to the present invention, also includes a radiation therapy agent and vaccines.

Sample

A sample may be, but is not limited to, a tissue section or biopsy, such as a portion of the neoplasm that is being treated or it may be a portion of the surrounding normal tissue. The sample may be but is not limited to blood, stool (faeces), urine, pleural fluid, gall, bronchial fluid, oral washings, tissue biopsies, ascites, pus, cerebrospinal fluid, follicular fluid, tissue or mucus. The sample may be processed prior to being assayed. For example, the sample may be diluted, concentrated or purified and/or at least one compound, such as an internal standard, may be added to the sample. The procedures for handling different samples are known the skilled artisan. Preferably, the sample is a tissue biopsy. Even more preferably the sample is a cancer tissue sample. Preferably the sample is from a human.

It is to be understood that the sample may have been obtained prior to the initiation of the methods according to the invention. Thus, the method may be performed without any interaction with the subject from which the sample has been obtained. Consequently, the method may be carried out in vitro.

Methods for Subtyping, Staging and Predict Risk of Developing a Cancer

The present invention relates to the identification of subtypes of cancers, which have up-regulated levels of the FcRn receptor. The surprising discovery of such subtypes are, by the inventing team, considered important from a clinical point of view since the presence of an up-regulated accessible receptor on cancer cells makes it an interesting target using FcRn binding agents coupled to a therapeutic drug, diagnostic agent or imaging agent.

Thus, in a first aspect, the invention relates to a method of subtyping a cancer (such as a malignant cancer), staging a cancer, and/or predicting the risk of developing a cancer, the method comprising
providing a biological sample (e.g. previously obtained) from a subject;
determining the level of FcRn in said sample; and
comparing said determined level to a reference level;
   wherein a higher level of FcRn in said sample compared to the reference level is indicative of an FcRn up-regulated subtype/stage; and wherein a level equal to or lower than said reference level is indicative of an FcRn normal subtype/stage or FcRn down-regulated subtype/stage;
   and/or
   wherein a higher level of FcRn in said sample compared to the reference level is indicative of an increased risk of developing a cancer; and wherein a level equal to or lower than said reference level is not indicative of an increased risk of developing a cancer. Phrased in another way, a level equal to or lower than said reference level is indicative of a normal sample. In Example 1, examples of cancer types with up-regulated FcRn levels are shown. In Examples 2-4, accumulation/targeting of engineered albumin high binders in human xenografts after intravenous injection in mice are shown.

Without being bound by theory, it is believed that similar subtypes may be identified in other cancer types, if larger cohorts are screened. Thus, the method according to the invention is relevant for subtyping and subsequent treatments of such cancer subtypes.

One technical advantage of an identified FcRn positive (up-regulated) subtype, is that such subtypes are likely treatable with an FcRn binding agent coupled to a therapeutic. Thus, in an embodiment, the method is for subtyping/identifying a cancer susceptible to treatment by an FcRn binding agent. In yet another embodiment, a higher level of FcRn in said sample compared to the reference level is indicative of a subtype/stage being susceptible to (improved) treatment by an FcRn binding agent. In the present context “improvement” is to be seen compared to a sample having a lower expression of FcRn.

As also mentioned above, it is believed that FcRn up-regulation may also be identified in other cancer types. Thus, in an embodiment, the cancer type is selected from the group consisting of lung cancer, pancreatic cancer, liver cancer, intestinal cancer, prostate cancer, bladder cancer, kidney cancer, ovarian cancer, cervical cancers, adenocarcinomas, squamous cell carcinomas, colorectal cancer, breast cancer, and ear nose throat cancers. In another embodiment, said cancer type is selected from the group consisting of breast cancer, colorectal cancer, lung cancers, pancreatic cancers, liver cancers, intestinal cancers, prostate cancers, bladder cancers, kidney cancers, such as renal clear cell carcinoma, ovarian cancers, cervical cancers, adenocarcinomas, squamous cell carcinomas, head and neck cancer and ear or nose or throat cancers. Example 6 shows overexpression in different cancer types compared to corresponding normal/healthy tissue.

In a preferred embodiment, the method is for subtyping a breast cancer or a colorectal cancer, the method comprising
providing a biological breast cancer sample or colorectal cancer sample (e.g. previously obtained) from a subject;
determining the level of FcRn in said sample; and
comparing said determined level to a reference level;

wherein a higher level of FcRn in said sample compared to the reference level is indicative of an FcRn up-regulated subtype; and wherein a level equal to or lower than said reference level is indicative of an FcRn normal or FcRn down-regulated subtype.

As mentioned above, it is believed that such FcRn up-regulated cancer subtypes are more susceptible to e.g. an albumin-facilitated treatment (see details below). Thus, in an embodiment, t the method is for subtyping/identifying a cancer susceptible to treatment by an FcRn binding agent (e.g. “albumin therapy”).

In Example 1, FcRn up-regulated subtypes have been identified in breast cancer tissue. Thus, in an embodiment said cancer type is breast cancer. Preferably, the method is for subtyping the cancer. In yet an embodiment the breast cancer is a Luminal B breast cancer or a Triple-negative/basal-like breast cancer.

In Example 1, FcRn up-regulated subtypes have also been identified in colorectal cancer tissue. Thus, in an embodiment said cancer type is colorectal cancer. Preferably, the method is for subtyping the cancer.

Cancer tissue may be benign or malignant. In addition, cancer types may be staged according to different staging protocols. In an embodiment, said sample is metastatic cancer tissue (non-benign), such as a metastatic tissue biopsy. In another embodiment, said sample is benign cancer tissue (non-malignant), such as a tissue biopsy. A stage may be considered a subtype according to the invention.

A reference level according to the invention may be selected from different types of reference levels normally employed by the skilled person in the field of cancer diagnosis/subtyping/staging. Thus, in an embodiment said reference level is the level of FcRn of a normal sample (non-cancer) of the same type or an average level from several normal samples. In yet another embodiment, said normal sample is from tissue bordering said biological sample, or tissue distant from said biological sample.

The source of the biological sample may be obtained from different sources. Thus, in a further embodiment, said biological sample is selected from the group consisting of tissue biopsies, blood, stool (faeces), urine, pleural fluid, saliva, gall, bronchial fluid, oral washings, ascites, pus, cerebrospinal fluid, follicular fluid, tissue and mucus, preferably a tissue biopsy. In Example 1, cancer tissue samples/biopsies are tested.

In relation to cancer subtyping, specific samples are preferred. Thus, in a further embodiment the biological sample is a cancer sample, such as a cancer tissue biopsy.

The FcRn level can be determined/established by different means. Thus, in an embodiment said level is determined using an FcRn binding agent (protein level). In yet another embodiment, said FcRn binding agent is selected from the group consisting of WT albumins, albumin variants, albumin binding agents, FcRn antibodies, IgG's, peptides or proteins, and nucleic acids, such as aptamers, preferably the binding agent is an albumin, even more preferably an albumin variant. In yet a preferred embodiment, said albumin variant has a higher binding affinity to FcRn than the WT version of albumin (SEQ ID NO: 1).

Relative to WT HSA, the variant may have a higher binding affinity to FcRn or a weaker binding affinity FcRn. Preferably the FcRn is human FcRn (hFcRn), more preferably soluble human FcRn (shFcRn). The albumin variant may be a naturally occurring variant or a recombinant variant.

The albumin variant may or may not comprise or consist of albumin domain III or variant thereof and at least one (e.g. several) additional albumin domain or fragment thereof, such as a second albumin domain III or a variant thereof, as disclosed in WO 2011/124718 (incorporated herein by reference). Suitably, the albumin variant comprises or consists of at least one (e.g. several) albumin domain III or variant or fragment thereof, wherein at least one (e.g. several) albumin domain III comprises one or more (e.g. several) substitutions in positions corresponding to the positions in SEQ ID NO: 1 selected among: 573, 500, 550, 417, 440, 464, 490, 492, 493, 494, 495, 496, 499, 501, 503, 504, 505, 506, 510, 535, 536, 537, 538, 540, 541, 542, 574, 575, 577, 578, 579, 580, 581, 582 and 584, as disclosed in WO 2011/051489 (incorporated herein by reference). Suitable substitutions include one or more (e.g. several) substitutions in positions corresponding to the positions in SEQ ID NO: 1 selected among: K573Y, W, P, H, F, V, I, T, N, S, G, M, C, A, E, Q, R, L, D, K500E, G, D, A, S, C, P, H, F, N, W, T, M, Y, V, Q, L, I, R, Q417A, H440A, H464Q, E492G, D494N,Q,A, E495Q,A, T496A, D494E+Q417H, D494N+T496A, E492G+V493P, P499A, E501A,Q, N503H,K, H510Q, H535Q, K536A, P537A, K538A, K541G,D, D550E,N, E492G+K573P,A, or E492G/N503H/K573P.

The albumin variant may comprise alterations at two or more (several) positions selected from positions corresponding to positions (a) 492 and 580; (b) 492 and 574; (c) 492 and 550; (d) 550 and 573; (e) 550 and 574; (f) 550 and 580 in SEQ ID NO: 1, as disclosed in WO 2014/072481 (incorporated herein by reference).

The albumin variant may comprise: (i) an N-terminal region comprising a first albumin which is a human albumin variant, in which the N-terminal of the first albumin comprises all amino acids of the human albumin variant except the C-terminal 2 to 30 amino acids; and (ii) a C-terminal region of a second albumin, which is selected from macaque albumin, mouse albumin, rabbit albumin, sheep albumin, human albumin, goat albumin, chimpanzee albumin, hamster albumin, guinea pig albumin, rat albumin, cow albumin, horse albumin, donkey albumin, dog albumin, chicken albumin, or pig albumin, or a variant thereof, in which the C-terminal of the second albumin or albumin variant comprises the C-terminal 2 to 30 amino acids of the second albumin or albumin variant; wherein the polypeptide has (i) an altered plasma half-life compared with the human albumin variant and/or (ii) an altered binding affinity to FcRn compared with the human albumin variant, as disclosed in WO 2012/059486 (incorporated herein by reference).

The albumin variant may comprise one or more (e.g. several) alterations in Domain I of the mature human albumin polypeptide sequence of SEQ ID NO: 1; and one or more (e.g. several) alterations in Domain III of the mature human albumin polypeptide sequence of SEQ ID NO: 1, wherein the one or more (e.g. several) alterations cause the polypeptide to have an altered binding affinity to FcRn, as disclosed in WO 2013/135896 (incorporated herein by reference). Suitably, the alteration(s) in Domain I are selected from positions corresponding to any of positions 78 to 120 of SEQ ID NO: 1, such as any of positions 78 to 88 and/or from any of 105 to 120; and the alteration(s) in Domain III are selected from positions corresponding to any of positions 425, 505, 510, 512, 524, 527, 531, 534, 569, 573, 575 of SEQ ID NO: 1. Suitably, the alteration at the position corresponding to positions 78 to 120 or 425, 505, 510, 512, 524, 527, 531, 534, 569, 573, and/or 575 of SEQ ID NO: 1 is a substitution; and the alteration is optionally a substitution selected from (i) 83N, K or S; (ii) 111D, G, H, R, Q or E; or (iii) 573P, Y, W, H, F, T, I or V.

The albumin variant may comprise one or more (e.g. several) alterations in Domain II of the mature human albumin polypeptide sequence of SEQ ID NO: 1 selected from the group consisting of positions corresponding to positions 349, 342, 381, 345, 384, 198, 206, 340, 341, 343, 344, 352, 382, 348, and/or 383 in SEQ ID NO: 1; wherein the one or more (e.g. several) alterations causes the albumin variant to have (i) an altered plasma half-life and/or (ii) an altered binding affinity to FcRn, as disclosed in WO 2015/036579 (incorporated herein by reference). Suitably, the alteration at the position corresponding to position 349, 342, 381, 345, 384, 198, 206, 340, 341, 343, 344, 352, 382, 348, and/or 383 is a substitution; and the alteration is optionally a substitution selected from (i) 349F, W, Y, H, P, K or Q, preferably F; (ii) 342Y, W, F, H, T, N, Q, A, C, I, L, P, V, preferably Y; (iii) 381G or A, preferably G; or (iv) 345E, H, I or Q.

The albumin variant may comprise a variant Domain III of an albumin, or fragment thereof, comprising a mutation, such as a substitution, corresponding to one or more (e.g. several) positions corresponding to V418, T420, V424, E505 and V547 of SEQ ID NO: 1. These mutations are disclosed in WO 2013/075066 (incorporated herein by reference). Substitutions may be at one, two or more (several, e.g. at two, three, four, or five) of the positions corresponding to V418, T420, V424, E505 and V547; for example, there may be one or more (e.g. several) substitutions selected from V418M, T420A, V424I, E505(R/K/G) and V547A. In a particular embodiment, the albumin comprises the substitutions V418M, T420A and E505R; or V418M, T420A, E505G and V547A. The albumin may comprise one or more (e.g. several) additional substitutions at positions selected from N429, M446, A449, T467, and A552; such as selected from N429D, M446V, A449V, T467M, and A552T.

The albumin variant may comprise a variant Domain III of an albumin, or fragment thereof, comprising one to eighteen amino acid substitutions to increase one or both of affinity for FcRn and serum half-life of the polypeptide, as disclosed in WO 2011/103076 (incorporated herein by reference). Substitutions may be at any one or more (e.g. several) of positions corresponding to positions 381, 383, 391, 401, 402, 407, 411, 413, 414, 415, 416, 424, 426, 434, 442, 445, 447, 450, 454, 455, 456, 457, 459, 463, 495, 506, 508, 509, 511, 512, 515, 516, 517, 519, 521, 523, 524, 525, 526, 527, 531, 535, 538, 539, 541, 557, 561, 566 or 569 of SEQ ID NO: 1. Suitable substitutions may be selected from V381N, V381Q, E383A, E383G, E383I, E383L, E383V, N391A, N391G, N391I, N391L, N391V, Y401D, Y401E, K402A, K402G, K4021, K402L, K402V, L407F, L407N, L407Q, L407W, L407Y, Y411Q, Y411N, K413C, K4135, K413T, K414S, K414T, V415C, V4155, V415T, Q416H, Q416P, V424A, V424G, V424I, V424L, V424N, V424Q, V426D, V426E, V426H, V426P, G434C, G434S, G434T, E442K, E442R, R445F, R445W, R445Y, P447S, P447T, E450D, E450E, S454C, S454M, 5454T, V455N, V455Q, V456N, V456Q, L457F, L457W, L457Y, Q459K, Q459R, L463N, L463Q, E495D, T506F, T506W, T506Y, T508K, T508R, T5085, F509C, F5091, F509L, F509M, F509V, F509W, F509Y, A511F, A511W, A511Y, D512F, D512W, D512Y, T515C, T515H, T515N, T515P, T515Q, T5155, L516F, L5165, L516T, L516W, L516Y, S517C, S517F, S517M, S517T, S517W, S517Y, K519A, K519G, K519I, K519L, K519V, R521F, R521W, R521Y, I523A, I523D, I523E, I523F, I523G, I523K, I523L, I523N, I523Q, I523R, I523V, I523W, I523Y, K524A, K524G, K524I, K524L, K524V, K525A, K525G, K525I, K525L, K525V, Q526C, Q526M, Q526S, Q526T, Q526Y, T527F, T527W, T527Y, E531A, E531G, E531I, E531L, E531V, H535D, H535E, H535P, K538F, K538W, K538Y, A539I, A539L, A539V, K541F, K541W, K541Y, K557A, K557G, K557I, K557L, K557V, A561F, A561W, A561Y, T566F, T566W, T566Y, A569H, and A569P; such as selected from L407N, L407Y, V415T, V424I, V424Q, V426E, V426H, P447S, V455N, V456N, L463N, E495D, T506Y, T508R, F509M, F509W, A511F, D512Y, T515Q, L516T, L516W, S517W, R521W, I523D, I523E, I523G, I523K, I523R, K524L, Q526M, T527Y, H535P and K557G.

The albumin variant may comprise a variant Domain III of an albumin, or fragment thereof, comprising amino acid substitutions at positions corresponding to the following positions of SEQ ID NO: 1: (a) residues 383 and 413; (b) residues 401 and 523; (c) residues 407 and 447; (d) residues 407 and 447 and 539; (e) residues 407 and 509; (f) residues 407 and 526; (g) residues 411 and 535; (h) residues 414 and 456; (i) residues 415 and 569; (j) residues 426 and 526; (k) residues 442 and 450 and 459; (l) residues 463 and 508; (m) residues 508 and 519 and 525; (n) residues 509 and 527; (o) residues 523 and 538; (p) residues 526 and 557; (q) residues 541 and 561; (r) residues 463 and 523; (s) residues 508 and 523; (t) residues 508 and 524; (u) residues 463, 508 and 523; (v) residues 463, 508 and 524; (w) residue 508, 523 and 524; (x) residue 463, 508, 523 and 524; (y) residues 463 and 524; (z) residues 523 and 524; and (aa) residues 463, 523, and 524, wherein the substitutions increase one or both of affinity for FcRn and serum half-life of the polypeptide, as disclosed in WO 2012/112188 (incorporated herein by reference). Suitable substitutions may be selected from (a) L463C, F, G, H, I, N, S or Q; (b) T508C, E, I, K, R or S; (c) I523A, C, D, E, F, G, H, I, K, L, M, N, P, Q, R, S, T, V, W or Y; (d) K524A, F, G, H, I, L, M, Q, T or V; (e) L463F or N; (f) T508R or S; (g) I523D, E, F, G, K or R; and (h) K524L.

The albumin variant may comprise one or more (e.g. several) alterations in the mature human albumin polypeptide sequence of SEQ ID NO: 1 selected from the group consisting of positions corresponding to positions V418, T420, V424, E505, V547, K573 in SEQ ID NO: 1; wherein the one or more (several) alterations causes the albumin variant to have (i) an altered plasma half-life and/or (ii) an altered binding affinity to FcRn.

The albumin variant may comprise one or more (e.g. several) alterations in the mature human albumin polypeptide sequence of SEQ ID NO: 1 selected from the group consisting of positions corresponding to positions V381, preferably V381N or Q; E383, preferably E383A, G, I, L, or V; N391, preferably N391A, G, I, L or V; Y401 preferably Y401D or E; K402, preferably K402A, G, I, L, or V; L407, preferably L407F, N, Q, W, or Y; Y411, preferably Y411Q, or N; K413, preferably K413C, S, or T; K414, preferably K414S or T; V415C, preferably V415C, S, or T; Q416, preferably Q416H or P; V424, preferably V424A, G, I, L, N, or Q; V426D, preferably V426D, E, H, or P; G434, preferably G434C, S, or T; E442, preferably E442K or R; R445, preferably R445F, W or Y; P447, preferably P447S or T; E450, preferably E450D or E; S454, preferably S454C, M or T; V455, preferably V455N or Q; V456, preferably V456N or Q; L457, preferably L457F, W or Y; Q459, preferably Q459K or R; L463, preferably L463N or Q; E495, preferably E495D; T506, preferably T506F, W or Y; T508, preferably T508K, R, or S; F509, preferably F509C, I, L, M, V, W or Y; A511, preferably A511F, W, or Y; D512, preferably D512F, W or Y; T515, preferably T515C, H, N, P, Q or S; L516, preferably L516F, S, T, W or Y; S517, preferably S517C, F, M, T, W or Y; K519, preferably K519A, G, I, L, or V; R521, preferably R521F, W or Y; 1523, preferably I523A, D, E, F, G, K, L, N, Q, R, V, W or Y; K524, preferably K524A, G, I, L or V; K525, preferably K525A, G, I, L or V; Q526, preferably Q526C, M, S, T or Y; T527, preferably T527F, W or Y; E531, preferably E531A, G, I, L or V; H535, preferably H535D, E or P; K538, preferably K538F, W or Y; A539, preferably A539I, L or V; K541, preferably, K541F, W or Y; K557, preferably K557A, G, I, L or V; A561, preferably A561F, W or Y; T566, preferably T566F, W or Y; A569, preferably A569H or P in SEQ ID NO: 1; wherein the one or more (e.g. several) alterations causes the albumin variant to have (i) an altered plasma half-life and/or (ii) an altered binding affinity to FcRn.

The albumin variant may comprise one or more (e.g. several) alterations in the mature human albumin polypeptide sequence of SEQ ID NO: 1 selected from the group consisting of positions corresponding to positions V547, preferably V457A; K573, preferably K573P or Y; 1523, preferably I523A or G, T527, preferably T527M, K500, preferably K500A; or E505, preferably E505Q in SEQ ID NO: 1; wherein the one or more (e.g. several) alterations causes the albumin variant to have (i) an altered plasma half-life and/or (ii) an altered binding affinity to FcRn.

The albumin variant may comprise one or more (e.g. several) alterations in the mature human albumin polypeptide sequence of SEQ ID NO: 1 selected from the group consisting of positions corresponding to positions 573, 523, 527 or 505 of SEQ ID NO: 1, preferably K573Y; I523G; I523A; T527M; E505Q; or K573P, for example K573Y and I523G; K573Y, I523G and T527M; K573Y, E505Q and T527M; K573Y and T527M; K573P and I523G; K573P, I523G and T527M; K573P, E505Q and T527M; K573P and T527M; V547A; V547A and K573P; V547A, E505Q, K573P and T527M; or K500A and H510Q of SEQ ID NO: 1.

The first binding agent can be detected in different ways, e.g. by using a sandwich assay employing a second binding agent binding to the first binding agent. The skilled person knows different ways of detecting surface molecules.

In an embodiment, the binding agent comprises a detectable label. In yet another embodiment the detectable label is chosen from radioisotopes, enzymes having detectable products, fluorophores, chemiluminescent compounds, magnetic particles, microparticles, microspheres, nanoparticles, nanospheres, biotin, streptavidin, and digoxin.

In certain cases, levels of FcRn may also be extrapolated from the expression level of FcRn (RNA). Thus, in an embodiment the FcRn level is determined by determining the RNA level of FcRn in the sample. The skilled person knows of methods for determining RNA levels. Examples of non-limiting methods are PCR (polymerase chain reaction), QPCR (quantitative PCR), FISH (fluorescence in situ hybridization), RCA (rolling circle amplification) etc.

In a certain embodiment, the method according the invention, further comprises administering and/or prescribing and/or recommending administration to a subject in need thereof a composition (for treatment of cancer) as defined according to the invention, if said subject is considered to have an FcRn up-regulated cancer subtype.

Composition for Use in Subtyping, Staging and Predict Risk of Developing a Cancer

A second aspect of the invention relates to a composition comprising an FcRn binding agent for use in the subtyping of a cancer, staging a cancer, and/or prediction of the risk of developing a cancer,

wherein a higher level of FcRn in a biological sample compared to a reference level is indicative of an FcRn up-regulated subtype/stage; and wherein a level equal to or lower than said reference level is indicative of an FcRn normal subtype/stage;
and/or
wherein a higher level of FcRn in said sample compared to the reference level is indicative of an increased risk of developing a cancer; and wherein a level equal to or lower than said reference level is not indicative of an increased risk of developing a cancer.

It is noted that embodiments of the first aspect of the invention can interchangeably be combined with this aspect.

In a preferred embodiment the composition is for use in the subtyping (diagnosis) of a colorectal cancer or a breast cancer,

wherein a higher level of FcRn in said sample compared to the reference level is indicative of an FcRn up-regulated subtype; and wherein a level equal to or lower than said reference level is indicative of an FcRn normal subtype.

As mentioned above, in an embodiment the composition is for subtyping/identifying a cancer susceptible to treatment by an FcRn binding agent.

In yet another embodiment, a higher level of FcRn in said sample compared to the reference level is indicative of a subtype/stage being susceptible to treatment by an FcRn binding agent.

Treatment of Cancers

As described above, the inventing team has surprisingly identified cancer subtypes, which express higher levels of the FcRn receptor. Such subtypes are considered relevant subtypes for treatments using FcRn binding agents.

Thus, a third aspect of the invention relates to a composition comprising an FcRn binding agent coupled to a therapeutic agent, for use in the treatment, prevention or alleviation of a cancer, wherein said cancer has up-regulated levels of FcRn compared to a reference level. Phrased in another way, the invention relates to a composition comprising an FcRn binding agent coupled to a therapeutic agent, for use in the treatment, prevention or alleviation of a cancer over-expressing FcRn. In another embodiment, said cancer type is selected from the group consisting of breast cancer, colorectal cancer, lung cancers, pancreatic cancers, liver cancers, intestinal cancers, prostate cancers, bladder cancers, kidney cancers, such as renal clear cell carcinoma, ovarian cancers, cervical cancers, adenocarcinomas, squamous cell carcinomas, head and neck cancer and ear or nose or throat cancers. Example 6 shows overexpression in different cancer types compared to corresponding normal healthy tissue. Example 8 shows that FcRn high-binders bind to (and/or get taken up by) FcRn expressing cells, whereas cells knocked-down for FcRn (FcRn negative cells) has much lower binding/uptake. In another embodiment, said cancer is a breast cancer and/or a colorectal cancer. Accumulation/targeting of engineered albumin high binders in human xenografts after intravenous injection in mice is shown in Examples 2-4 and 7. Thus, Alexa Fluor 680 labelled Albumin variants were used as a model system for verification of targeting/accumulation of FcRn binders coupled to drugs and/or imaging agents in vivo.

The FcRn binding agent can be coupled to the therapeutic agent in different ways. Thus, in an embodiment, the FcRn binding agent is coupled to the therapeutic agent by fusion, conjugation or association.

In yet another embodiment, said FcRn binding agent is selected from the group consisting of WT albumins, albumin variants, FcRn antibodies, IgG's, peptides or proteins, and nucleic acids, such as aptamers, preferably the binding agent is an albumin, even more preferably an albumin variant. In yet another embodiment, the variant HSA has one or more (several) improved pharmacokinetic properties when compared with wild type has for example altered half-life such as increased or decreased half-life. In yet another embodiment, the variant HSA has a higher binding to FcRn and/or a longer half-life than wild type HSA.

In an embodiment, said albumin variant has a higher binding affinity to FcRn than the WT version of albumin (SEQ ID NO: 1). In a preferred embodiment, the variant is SEQ ID NO: 4 or SEQ ID NO: 5.

Different therapeutic agents may be fused/conjugated/associated to the FcRn binding agent. Thus, in an embodiment, the therapeutic agent is selected from the group consisting of a radionuclide, an anti-cancer drug, such as Actinomycin-D, Aldesleukin, Alemtuzumab, alkane sulfonates, Alkeran, Amsacrine, Anastrozole, Anastrozole, anthracyclines, antimetabolites, Ara-C, Arsenic trioxide, Asparaginase, Azathioprine, BCG, Bicalutamide, BiCNU, Bleomycin, Bortezomib, Busulfan, Busulphan, Capecitabine, Carboplatin, Carboplatinum, Carmustine, CCNU, Cetuximab, Chlorambucil, Chloramphenicol, chorionic, Ciclosporin, Cidofovir, Cisplatin, Cladribine, Coal tar containing products, Colchicine, CPT-11, Cyclophosphamide, Cytarabine, Cytosine arabinoside, Cytoxan, Dacarbazine, Dactinomycin, Danazol, Dasatinib, Daunorubicin, Dexrazoxane, Diethylstilbestrol, Dinoprostone, Dithranol containing products, Docetaxel, Doxorubicin, DTIC, Dutasteride, Epirubicin, Estradiol, Estramustine, Ethyleneimine, Etoposide, Exemestane, Finasteride, Floxuridine, Fludarabine, Fluorouracil, Flutamide, folate analogs, Fotemustine, Ganciclovir, Gemcitabine, Gemtuzumab, Gonadotrophin, Goserelin, Herceptin, Hexamethylamine, hormonal agents, Hydroxycarbamide, Hydroxyurea, Idarubicin, Ifosfamide, Imatinib mesylate, Interferon containing products (including peginterferon), Irinotecan, Leflunomide, Letrozole, Leuprorelin acetate, Lomustine, Mechlorethamine, Medroxyprogesterone, Megestrol, Melphalan, Menotropins, Mercaptopurine, Methotrexate, Mifepristone, Mitomycin, Mitotane, Mitoxantrone, Methotrexate (MTX), Mycophenolate mofetil, Nafarelin, nitrogen mustards, nitrosorueas, Oestrogen containing products, Oxaliplatin, Oxytocin, Paclitaxel, Pamidronate, Pentamidine, Pentostatin, platinum compounds, Plicamycin, Podophyllyn, Procarbazine, Progesterone containing products, purine analogs, pyrimidine analogs, Raloxifene, Raltitrexed, Ribavarin, Rituximab, Sirolimus, Steroids, STI-571, Streptozocin, syntocinon, syntometrine, Tacrolimus, Tamoxifen, taxanes, Temozolomide, Teniposide, Testosterone, Tetrazine, Thalidomide, Thioguanine, Thiotepa, Tomudex, topoisomerase inhibitors, Topotecan, Toremifene, Trastuzumab, Treosulphan, Trifluridine, Trimetrexate, Triptorelin, Valganciclovir, Vidaradine, Vinblastine, vinca alkaloids, Vincristine, Vindesine, Vinorelbine, VP-16, Xeloda, and Zidovudine.

In yet an embodiment, the FcRn binding agent is further coupled to an imaging agent as described in further detail below.

In Vivo Imaging of Cancer

As described above, the inventing team has surprisingly identified cancer subtypes, which express higher levels of the FcRn receptor compared to normal tissue of the same type. Such subtypes may be imaged in vivo using FcRn targeting/binding agents coupled to a detectable moiety.

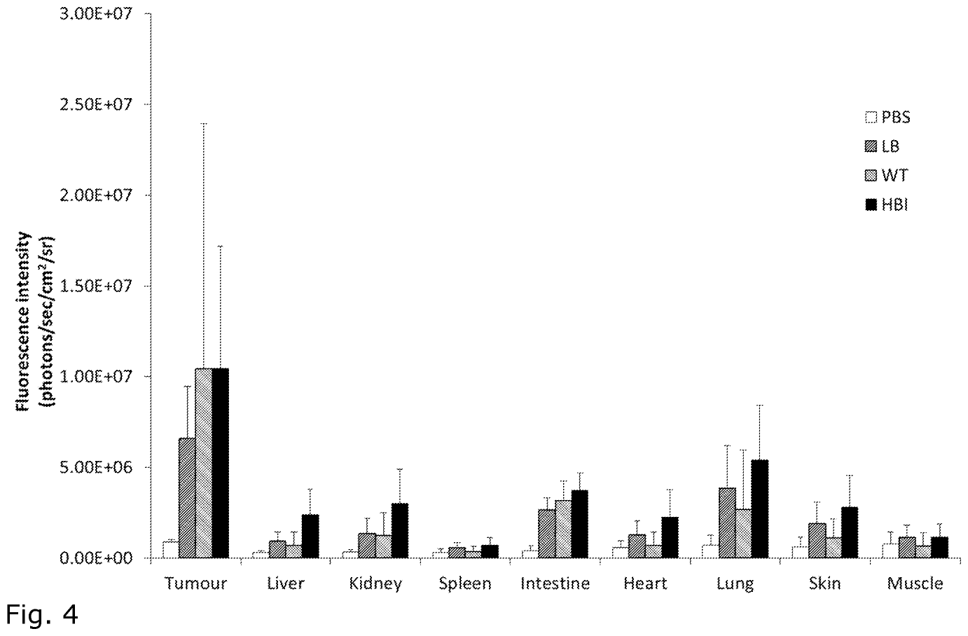

Thus, a further aspect of the invention relates to a composition comprising an FcRn binding agent coupled to a detectable moiety (imaging moiety) for use in the in vivo imaging of a cancer, wherein said cancer has up-regulated levels of FcRn compared to a reference level. Phrased in another way, the invention relates to a composition comprising an FcRn binding agent coupled to an imaging agent, for use in the in vivo imaging of a cancer over-expressing FcRn. Examples 3, 4 and 7 (and corresponding FIGS. 4-14+17) present in vivo imaging data of mice.

Thus, in a more specific aspect the invention relates to a method of obtaining an image of a (FcRn positive/FcRn upregulated) cancer (in vivo) in a subject, the method comprising the steps of:
a) delivering to a subject animal or human a pharmaceutically acceptable composition comprising FcRn binding agent coupled to a detectable moiety;
b) imaging the subject animal or human to identify a detectable signal from the FcRn binding agent coupled to a detectable moiety in the subject; and
c) generating an image of the detectable signal, thereby obtaining an image of a (FcRn positive/FcRn upregulated) cancer in the subject animal or human.

In yet an aspect the invention relates to an FcRn binding agent coupled to a detectable moiety for use in a method for diagnosing/imaging of a (FcRn positive) cancer (in vivo) in a subject, said method comprising:
a) administering to a subject said FcRn binding agent coupled to a detectable moiety, and
b) imaging the subject to detect said FcRn binding agent that is coupled to a detectable moiety that binds to the (FcRn positive/FcRn upregulated) cancer.

In a special embodiment, it may be a radioactive detectable moiety for imaging useful for PET or SPECT. In another embodiment it may be a non-radioactive detectable moiety useful for imaging, e.g., optical or MRI.

Various radiolabelled compositions have been developed for site-specific targeting of various antigens for SPECT and PET imaging. The general principle involves attaching a (positron) emitting radionuclide to a peptide and/or protein having a high specificity for a particular antigen, to visualize and quantify the expressing level using SPECT and PET imaging. This field of research has shown particular applicability for tumor diagnosis, staging and treatment monitoring. In an embodiment, the radionuclide is selected from the group consisting of ¹¹C, ¹⁵O, ¹⁸F labelled fludeoxyglucose, ⁶⁴Cu, ⁶⁸Ga, ⁶⁶Ga, ⁶⁰Cu, ⁶¹Cu, ⁶²Cu, ⁸⁹Zr, ¹²⁴I, ⁷⁶Br, ⁸⁶Y, ^{94m}Tc, ¹³¹I, ^{G67}Ga, ¹¹¹In, ¹²³I, and ^{99m}Tc.

In an embodiment, especially suited for MRI, paramagnetic agents such as gadolinium chelates, and superparamagnetic iron oxide particles may be used. In an embodiment, especially suited for optical imaging, quantum dots, fluorescence and bioluminescence agents, and FRET molecules may be used.

In yet an embodiment, the cancer to be visualized in vivo, is selected from the group consisting of lung cancers, pancreatic cancers, liver cancers, intestinal cancers, prostate cancers, bladder cancers, kidney cancers, such as renal clear cell carcinoma, ovarian cancers, cervical cancers, adenocarcinomas, squamous cell carcinomas, colorectal cancers, breast cancers, head and neck cancer and ear or nose or throat cancers.

In yet an embodiment, the subject is a human or animal, preferably a human.

Preferably, the FcRn binding agent is a high binding albumin as described in here.

As mentioned in different aspects above, the composition according to the invention may be used to treat e.g. cancers or to image a cancer. In fact these aspects can be combined into what is also called a “theranostics”, meaning that the composition may (simultaneously) be used for treatment and imaging. Thus, in an aspect the invention relates a composition comprising an FcRn binding agent coupled to one or more theranostic agent. This may be done by coupling different agent to the FcRn binding molecule, such as a detectable moiety+ a therapeutic agent. Thus, in an embodiment the FcRn binding molecule comprises both a detectable moiety and a therapeutic agent. In a further embodiment the detectable moiety and the therapeutic agent is the same molecule, such as a radionuclide. Theranostics is an emerging field especially within the field of personalized medicine.

Identification of Subtypes of Inflammatory Diseases

Besides the identification of cancer subtypes over-expressing FcRn, the inventing team has also identified such subtypes among inflammatory diseases. Such subtypes are considered important from a clinical point of view, since the presence of an up-regulated accessible (surface) receptor on inflammatory cells and/or inflamed cells, makes it an interesting target using FcRn binding agents coupled to a therapeutic drug, diagnostic agent or imaging agent. Example 5 shows the presence of FcRn in inflammatory disease (rheumatoid arthritis (RA)).

Thus, an aspect of the invention relates to a method of subtyping an inflammatory disease, the method comprising
providing a biological sample (e.g. previously obtained) from a subject;
determining the level of FcRn in said sample; and
comparing said determined level to a reference level;
   wherein a higher level of FcRn in said sample compared to the reference level is indicative of an FcRn up-regulated subtype; and wherein a level equal to or lower than said reference level is indicative of an FcRn normal subtype or an FcRn down-regulated subtype.

In an embodiment, said sample type is selected from the group consisting of biopsies, such as joint biopsies, e.g. from a knee joint, an elbow joint or a finger joint. In a more specific embodiment, the tissue is synovial tissue.

In a certain embodiment the method according the invention further comprises administering and/or prescribing and/or recommending administration to a subject in need thereof a composition (e.g. for treatment of inflammatory diseases) as defined according to the invention if said subject is considered to have an FcRn up-regulated inflammatory disease subtype.

Composition for Use in Subtyping or Staging an Inflammatory Disease

A further aspect of the invention relates to a composition comprising an FcRn binding agent for use in the subtyping or staging of an inflammatory disease, wherein a higher level of FcRn in a biological sample compared to a reference level is indicative of an FcRn up-regulated subtype/stage; and wherein a level equal to or lower than said reference level is indicative of an FcRn normal subtype/stage.

It is again noted that embodiments of the previous aspects of the invention can interchangeably be combined with this aspect.

Treatment of Inflammatory Diseases

As also described above, inflammatory disease subtypes, which expressing accessible FcRn receptor (e.g. on their surface) has been identified. Such subtypes are considered relevant subtypes for treatment using FcRn binding agents.

Thus, yet an aspect of the invention relates to a composition comprising an FcRn binding agent coupled to an anti-inflammatory drug, e.g. for use in the treatment of inflammatory diseases;

wherein said inflammatory disease has up-regulated levels of the FcRn receptor.

In an embodiment said inflammatory disease or disorder is selected from the group consisting of arthritis, asthma, ulcerative colitis, inflammatory bowel syndrome, allergies, allergic rhinitis/sinusitis, skin allergies, urticaria, angioedema, atopic dermatitis, food allergies, drug allergies, insect allergies, mastocytosis, osteoarthritis, rheumatoid arthritis, spondyloarthropathies, cardiovascular disease with an inflammation-based etiology, arterial sclerosis, transplant rejection, and graft versus host disease, preferably arthritis.

In yet another embodiment, said anti-inflammatory drug is selected from the group consisting of a NSAID's substance, such as selected from the group consisting of lornoxicam, diclofenac, nimesulide, ibuprofen, piroxicam, piroxicam (betacyclodextrin), naproxen, ketoprofen, tenoxicam, aceclofenac, indometacin, nabumetone, acemetacin, morniflumate, meloxicam, flurbiprofen, tiaprofenic acid, proglumetacin, mefenamic acid, fenbufen, etodolac, tolfenamic acid, sulindac, phenylbutazone, fenoprofen, tolmetin, acetylsalicylic acid, dexibuprofen, Cytokine blockers e.g. TNFα and pharmaceutically acceptable salts, complexes and/or prodrugs thereof and mixtures thereof.

In yet another embodiment, the compositions for use according to the invention may further comprise a pharmaceutically acceptable carrier and/or diluent.

In Vivo Imaging of Inflammatory Diseases, Such as Rheumatoid Arthritis

As described above, the inventing team has surprisingly also identified inflammatory diseases (such as rheumatoid arthritis) subtypes, which express higher levels of the FcRn receptor compared to normal tissue of the same type. Such subtypes may be imaged in vivo using FcRn binding agents coupled to a detectable moiety.

Thus, a further aspect of the invention relates to a composition comprising an FcRn binding agent coupled to a detectable moiety (imaging moiety) for use in the in vivo imaging of a inflammatory diseases, such as rheumatoid arthritis, wherein said inflammatory diseases (such as rheumatoid arthritis) has up-regulated levels of FcRn compared to a reference level. Phrased in another way, the invention relates to a composition comprising an FcRn binding agent coupled to an imaging agent, for use in the in vivo imaging of an inflammatory disease (such as rheumatoid arthritis) over-expressing FcRn.

Thus, in a more specific aspect the invention relates to a method of obtaining an image of an FcRn positive (and/or FcRn upregulated) inflammatory disease, such as rheumatoid arthritis, (in vivo) in a subject, the method comprising the steps of:
a) delivering to the subject animal or human a pharmaceutically acceptable composition comprising FcRn binding agent coupled to a detectable moiety;
b) imaging the subject animal or human to identify a detectable signal from the FcRn binding agent coupled to a detectable moiety in the subject; and
c) generating an image of the detectable signal, thereby obtaining an image of the FcRn positive (and/or FcRn upregulated) inflammatory disease (such as rheumatoid arthritis) in the subject animal or human.

In yet an aspect the invention relates to an FcRn binding agent coupled to a detectable moiety for use in a method for diagnosing/imaging of an FcRn positive (and/or FcRn upregulated) inflammatory diseases, such as rheumatoid arthritis (in vivo) in a subject, said method comprising:
a) administering to the subject said FcRn binding agent coupled to a detectable moiety, and
b) imaging the subject to detect said FcRn binding agent coupled to a detectable moiety bound to the (FcRn positive/upregulated) inflammatory disease.

In a special embodiment, it may be a radioactive detectable moiety for imaging useful for PET or SPECT. In another embodiment, it may be a non-radioactive detectable moiety useful for imaging, e.g., optical or MRI.

Various radiolabelled compositions have been developed for site-specific targeting of various antigens for SPECT and PET imaging. The general principle involves attaching a (positron) emitting radionuclide to a peptide and/or protein having a high specificity for a particular antigen, to visualize and quantify the expressing level using SPECT and PET imaging. This field of research has shown particular applicability for tumor diagnosis, staging and treatment monitoring. In an embodiment, the radionuclide is selected from the group consisting of ¹¹C, ¹⁵O, ¹⁸F labelled fludeoxyglucose, ⁶⁴Cu, ⁶⁸Ga, ⁶⁶Ga, ⁶⁰Cu, ⁶¹Cu, ⁶²Cu, ⁸⁹Zr, ¹²⁴I, ⁷⁶Br, ⁸⁶Y, ^{94m}Tc, ¹³¹I, ^{G67}Ga, ¹¹¹In, ¹²³I, and ^{99m}Tc.

In an embodiment, especially suited for MRI, paramagnetic agents such as gadolinium chelates, and superparamagnetic iron oxide particles may be used. In an embodiment, especially suited for optical imaging, quantum dots, fluorescence and bioluminescence agents, and FRET molecules may be used.

It should be noted that embodiments and features described in the context of one of the aspects of the present invention also apply to the other aspects of the invention.

All patent and non-patent references cited in the present application, are hereby incorporated by reference in their entirety.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. Furthermore, individual features mentioned in different claims, may possibly be advantageously combined, and the mentioning of these features in different claims does not exclude that a combination of features is not possible and advantageous.

The invention will now be described in further details in the following non-limiting examples.

### EXAMPLES

### Example 1

FcRn Expression in Patient Biopsies

Aim of Study

To identify FcRn levels in cancer and healthy tissue.

Materials and Methods

Materials

Two different cancer types were investigated (5 samples per cancer type). These were colon and breast cancer. Healthy bordering tissue was used as control.

The human cancer tissue samples were provided by The Pathology Institute, Aarhus University Hospital, 8000 Aarhus C, Denmark.

Immunohistochemical analysis of the human cancer tissue samples Human tissue was formalin fixed and paraffin embedded (FFPE). FFPE human tissue sections were treated with Tissue Clear Xylene substitute (Tissue-Tek/Sakura Finetek) to de-paraffinise slides. Next, slides were rehydrated by gradually decreasing ethanol solutions from 100% to 75% and finally moved to running cold tap water.

Antigen retrieval was performed in citrate buffer pH 6.0 by heating in a microwave oven at 800 W for 8 min, followed by 560 W for 2×14 min and finally cooling for 20 min.

The staining procedure was performed on an Autostainer Link 48 Instrument (Dako). The slides were blocked using protein block (Dako) and stained with the primary polyclonal rabbit human FCGRT antibody (HPA012122, Sigma) in dilution 1:200.

For visualization the Dako EnVision™ FLEX (kit K8023) detection system was used. This included endogenous peroxidase blocking containing hydrogen peroxidase (EnVision™ FLEX Peroxidase-blocking Reagent, SM801), a polymer coupled with Horseradish Peroxidase (HRP) and goat secondary antibody against rabbit immunoglobulins (EnVision™ FLEX/HRP, SM802), a DAB chromogen (EnVision™ FLEX DAB+ Chromogen, DM827) and finally hematoxylin (EnVision™ FLEX Hematoxylin, K8008).

Results

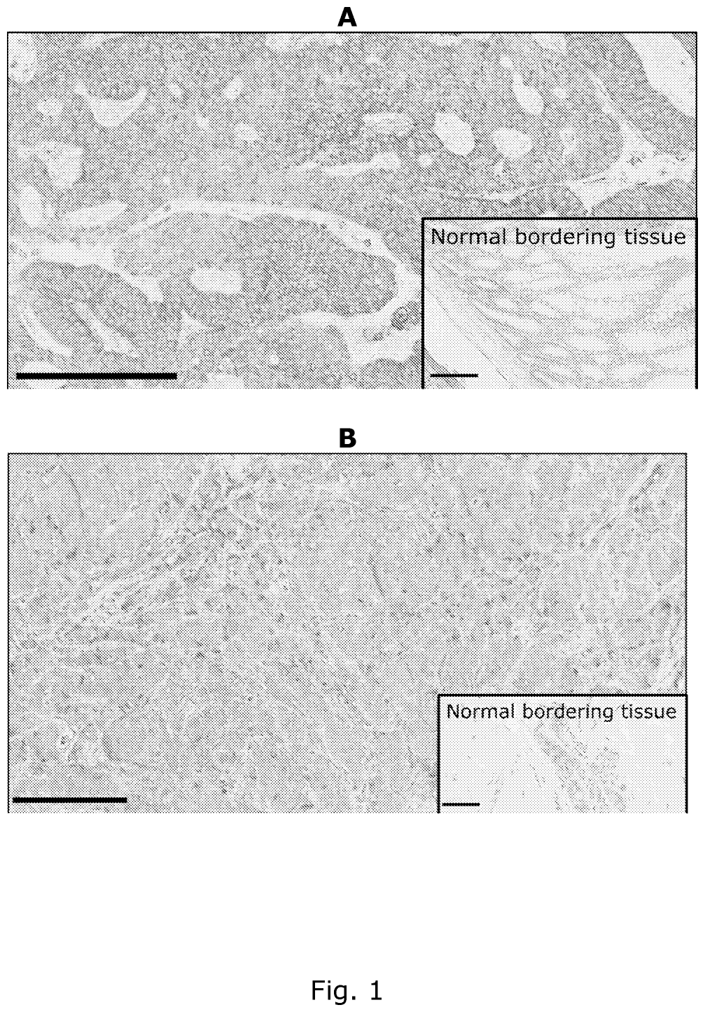

The results presented in FIG. 1 show representative examples of a breast cancer subtype and a colon cancer subtype, which have been identified showing higher expression levels of FcRn compared to corresponding healthy tissue.

Conclusion

It is been found that breast cancers and colon cancers over-expressing the FcRn receptor exist. It is believed that when such subtypes have been identified, the FcRn receptor could also be used as a binding agent for targeted cancer treatment of these specific subtypes.

### Example 2

FcRn expression in human cancer cell lines mouse xenografts

Aim of Study

Identification of FcRn overexpression in human cancer xenografts in mice.

Materials and Methods

Cancer Models

PXBC-3 pancreatic cancer cell

HT-29 human colorectal

Adenocarcinoma cells

MCF-7 human breast cancer cells

Results

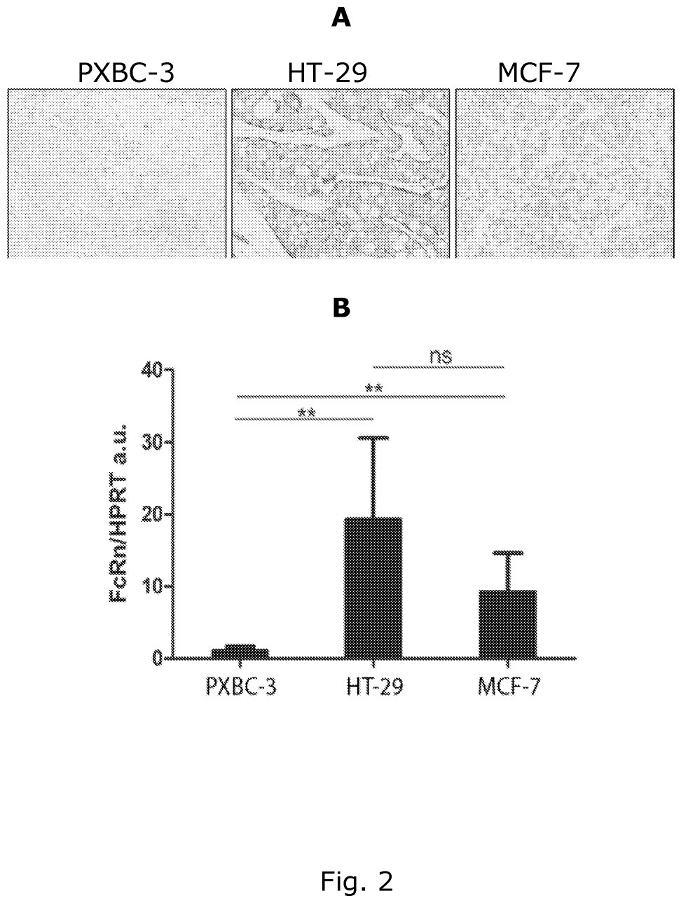

The results presented in FIG. 2 show higher FcRn expression in human colorectal adenocarcinoma cells (HT-29) and human breast cancer cells (MCF-7) compared to PXBC-3 pancreatic cancer cells.

Conclusion

FcRn overexpressing cancers can be identified in mice xenografts that can be used for in vivo FcRn binding agent experiments or cancer treatment.

### Example 3

Accumulation/targeting of engineered albumin high binders in human xenografts after intravenous injection in mice. Xenograft model 1: Bioluminescent xenografts.

Aim of Study

To investigate biodistribution and half-life, and tumour accumulation of albumin variants.

Materials and Methods

Albumin Variants

Alexa Fluor 680 labelled engineered albumin variants were provided by Albumedix Ltd. (Nottingham, UK).
HSA-K573P (High-binder I) (HBI) (SEQ ID NO: 4)
HSA-K500A (Low-binder) (LB) (SEQ ID NO: 6)

In Vivo Studies

All animal experiments were performed at the Animal Facility at the Institute of Biomedicine at Health, Aarhus University. All experiments were approved by the Danish Experimental Animal Inspectorate.

Cell Culture

The human cancer cell line (MDA-MB-231/Luc), a luciferase-expressing breast cancer cell line, was used in this study. Cells were maintained in DMEM (4500 mg/L D-glucose, L-glutamine) (Gibco, Life Technologies, #41965-039) with 10% fetal bovine serum (FBS) (Gibco, Life Technologies, #10270-106), 0.1 mM MEM Non-essential amino acids (Gibco, Life Technologies, #11140-035), 2 mM L-glutamine (Lonza, #BE17-605E) and 1% penicillin and streptomycin (Gibco, Life Technologies, #15140-122).

Bioluminescent Tumour Xenografts

Breast cancer MDA-MB231/Luc cells (4×10⁶ cells in 400 μl of 1:1 solution of PBS and matrigel, Geltrex™ LDEV-Free Reduced Growth Factor Basement Membrane Matrix, (Gibco, Life Technologies)) were injected subcutaneously into the right flank of each mouse (11 weeks old, female, Balb/canRj-Foxn1-nu, Janvier). Isoflurane was used as anesthesia for inoculation of tumour cells. Tumours were allowed to grow until visible (11 days) before they were randomly allocated into four group with approximately the same total tumour volume in each group (N=4 in treatment groups, N=3 in control group). Treatment was performed with 10 mg/kg fluorescent albumin for treatment groups and 150 μl PBS for the control group. Tumour sizes were measured on the days following the treatment by caliper measurements using the largest longitudinal (length) and width diameter of the tumour. The tumour volume was calculated by the formula (tumour volume=½ (length×width²).

At the end of the experimental time period, the mice were killed by cervical dislocation when anesthetized and the tumours and organs were collected. Blood and organs were analysed using the IVIS® Spectrum Bioimager (PerkinElmer, Waltham, Mass.). Background autofluorescence was eliminated using spectral unmixing and subsequent data analysis was carried out using Living Image software, version 4.3.1 (PerkinElmer).

Tumours, liver and kidney were snap-frozen for RNA isolation and fixed in neutral buffered formalin (10%) for preservation for immunohistochemistry studies. After 72 hours the tissue was dehydrated through a series of graded ethanol baths to displace water and next infiltrated with wax. The infiltrated tissue was then embedded into wax blocks.

Imaging and Quantification of Bioluminescence Data

The mice were injected subcutaneously with luciferin (D-luciferin, Caliper Life Sciences, Hopkinton, Mass.) at 300 mg/kg mouse body weight before being anesthetized with 3.5% isoflurane. Ten minutes after D-luciferin injection a whole body scan was performed with a Xenogen IVIS Spectrum imaging platform (PerkinElmer, MA), continuously applying 3.5% isoflurane anesthesia. A region of interest (ROI) was manually selected over relevant regions. The measured intensity was given as surface radiance (photons/s/cm2/sr) within a ROI

Termination of the experiment was performed by cervical dislocation ten minutes after D-luciferin injection to ensure allowance of assessment of ex vivo bioluminescence. The tumours and organs of interest were collected and ex vivo imaged with the IVIS scanner within 40 minutes.

Albumin Injection and Sample Preparation

Alexa Fluor 680 labelled Albumin variants were injected intravenously at 2 mg/ml in the tail. After injection, blood samples were taken from the tongue (1 minute sample) and by tail nicking at 4 hours, 24 hours, 48 hours and 72 hours into heparin coated capillary tubes (Hirschmann® Laborgerate GmbH & Co.). The samples were transferred into microfuge tubes and centrifuged to fraction blood cells from serum. Samples were stored at 4° C. until scanning was performed.

Results

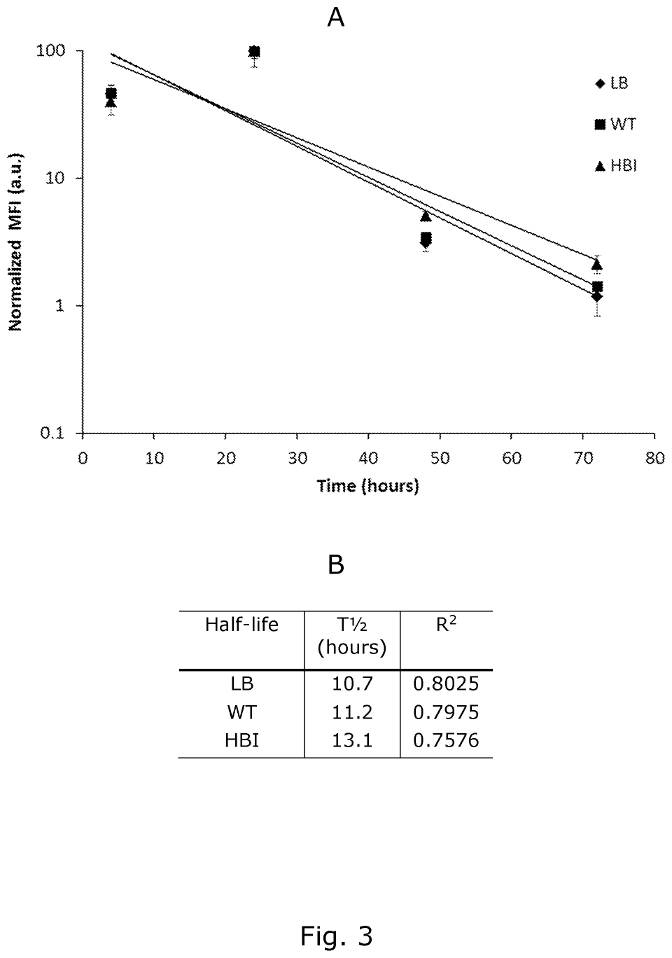

The results depicted in FIG. 3A show the half-life of albumin variants and the corresponding exponential trend lines. It is observed that the trend line is steepest for the low-binder and levels out more for the high-binder, meaning a higher half-life for the high-binder. This is also shown in FIG. 3B with the calculated half-life values for all albumin types. The longest half-life is observed for the high-binder.

The results shown in FIG. 4 show the fluorescence intensity of the organs and tumours ex vivo with the entire organ and tumour chosen. The highest fluorescence is observed in the tumour compared to other organs for all albumin variants.

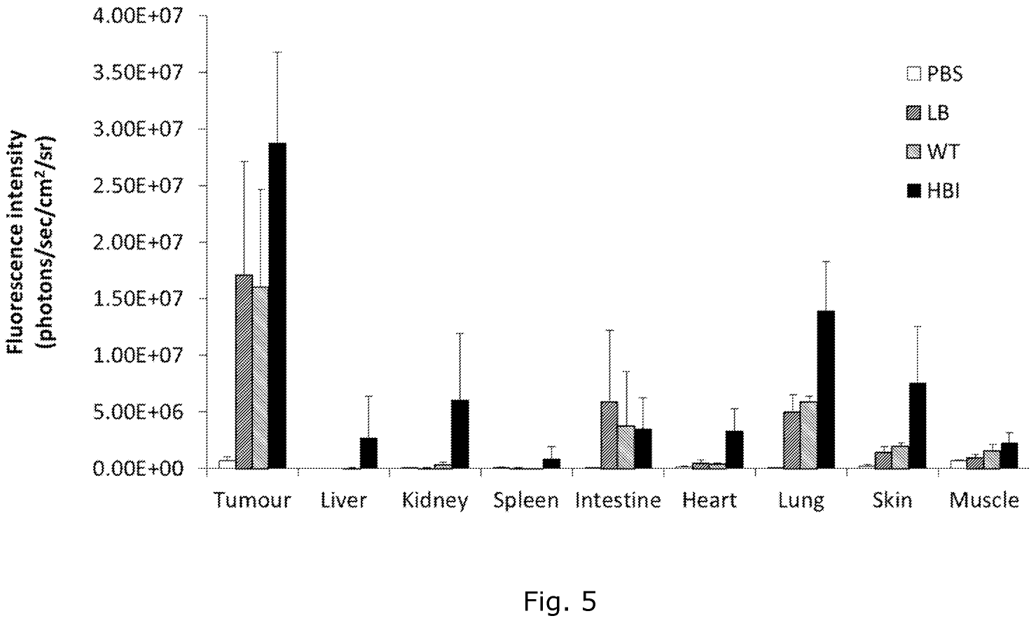

For an alternative method of comparison, a constant region of interest was selected for all organs and tumours and the result is shown in FIG. 5. Here it is clearly observed that the albumin variants accumulate in the tumours compared to organs and with the high-binder to the highest degree.

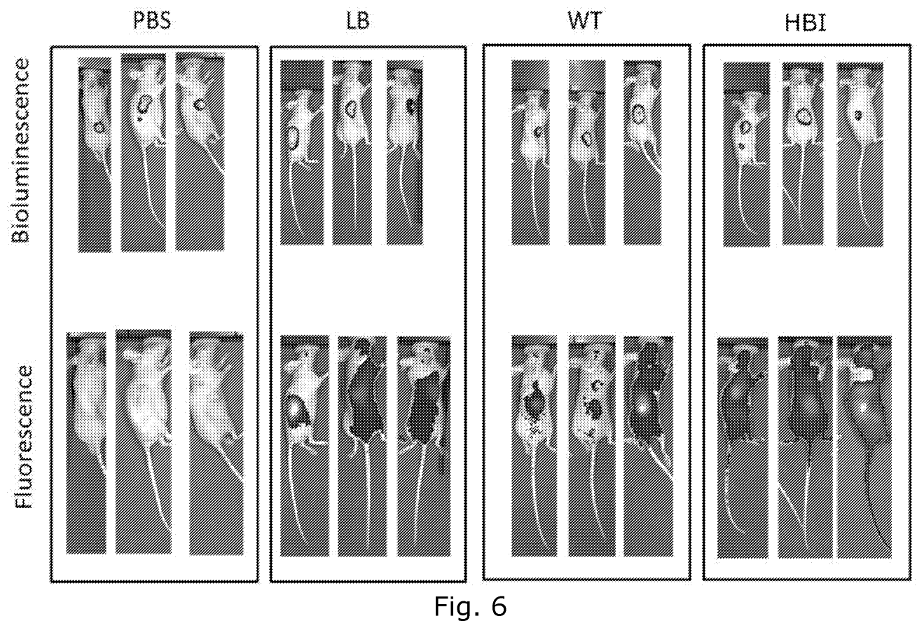

FIG. 6 shows the localization of the tumours by measuring the bioluminescence (top panel) which matches the presence of the albumin variants measured by fluorescence after spectral unmixing (lower panel) giving a visual indication of accumulation of albumin variants in tumours.

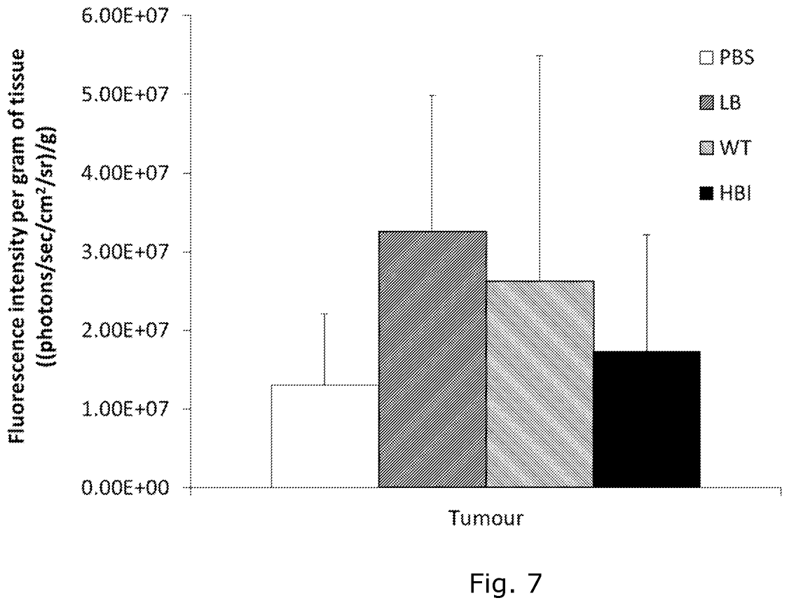

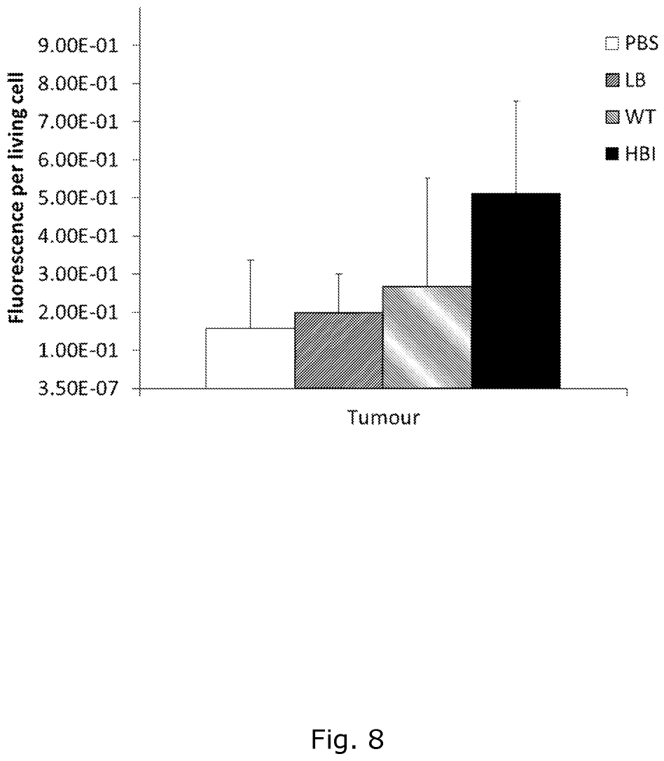

The results presented in FIG. 7 shows the fluorescence intensity of the albumin variants adjusted to the weight of the tumour. As the tumours can also consist of necrosis and edema tissue, adjustment to luciferase-expressing living tumour cells was performed by using the bioluminescence depicted in FIG. 8. Here it is observed that the high-binder albumin variant accumulates more than the other albumin variants.

Conclusion

Highest accumulation in tumors is observed with the high-binder albumin variant when correcting for living tumor cells using bioluminescence.

### Example 4

Accumulation/targeting of engineered albumin high binders in human xenografts after intravenous injection in mice. Xenograft model 2: Breast cancer xenograft (Non-luminescent cells)

Aim of Study

To investigate biodistribution and half-life of albumin in non-luminescent tumor xenografts.

Materials and Methods

Albumin Variants

Alexa Fluor 680 labelled engineered albumin variants were provided by Albumedix Ltd.
HSA-E492G, K573P, K574H, Q580K (High-binder II) (HBII) (SEQ ID NO: 5)
HSA-K500A (Low-binder) (LB) (SEQ ID NO: 6)

In Vivo Studies

All animal experiments were performed at the Animal Facility at the Institute of Biomedicine at Health, Aarhus University. All experiments were approved by the Danish Experimental Animal Inspectorate.

Cell Culture

The human breast cancer cell line MCF-7 was cultured at 37° C. in a humidified air atmosphere with 5% CO₂. The MCF-7 cells were grown in DMEM (Gibco, Life Technologies, #61965-026) medium supplemented with 10% fetal bovine serum (Gibco, Life Technologies, #10270-106), 1% penicillin/streptomycin (Gibco, Life Technologies, #15140-122) and 10 ug/ml insulin (Sigma, #I9278).

Human Tumour Xenograft

At 3 days before cell inoculation, a 3-estradiol pellet (0.5 mg, 60 days release; Innovative Research of America, Sarasota, Fla., USA) was implanted subcutaneously in the neck region of the mice to support tumour growth. MCF-7 cells (7.5×10⁶ cells in 400 μl of 1:1 solution of PBS and matrigel, Geltrex™ LDEV-Free Reduced Growth Factor Basement Membrane Matrix, (Gibco, Life Technologies)) were injected subcutaneously into the right flank of each mouse (11-16 weeks old, female, Balb/cAnRj-Foxn1-nu, Janvier). Isoflurane was used for anesthesia for implementation of pellets and inoculation of tumour cells. Tumour sizes were measured 2-3 times a week by caliper measurements of the length and width, and the tumour volume calculated. After 15 days of tumour growth injection of albumin variants was performed. Beforehand the mice were assigned to different treatment groups by randomly allocating the mice so each group had approximately the same tumour volume (tumour volume=n/6×L×W²). (HBII, N=7; WT, N=6, LB N=6, PBS N=3).

The experiment was terminated by cervical dislocation of the mice, organs and tumours were removed, scanned in the IVIS scanner and snap-frozen for RNA isolation and/or stored in 10% neutral buffered saline for histology.

Albumin Injection and Sample Preparation

Alexa Fluor 680 labelled Albumin variants were injected intravenously at 2 mg/ml in the tail. After injection, blood samples were taken from the tongue (1 minute sample) and by tail nicking at 4 hours, 24 hours, 48 hours and 72 hours into heparin coated capillary tubes (Hirschmann® Laborgerate GmbH & Co.). The samples were transferred into microfuge tubes and centrifuged to fraction blood cells from serum. Samples were stored at 4° C. until scanning was performed.

Imaging and Quantification of Fluorescence Data

Mice were anesthetized continuously with 3.5% isoflurane and a full body scan was performed in a Xenogen IVIS Spectrum imaging platform (PerkinElmer, MA). Organs and tumours were scanned ex vivo in the IVIS scanner. Spectral unmixing measurement was performed using one excitation wavelength (ex: 675 nm) and measuring at four different emission wavelengths (emission: 720 nm, 740 nm, 760 nm, 780 nm).

The images were loaded as a group in the Living Image 4.3.1 software for comparison and an ROI was manually selected over relevant regions. The measured intensity was given as surface average radiance (photons/s/cm²/sr). Spectral unmixing was not performed for the full body scans at 4 and 21 hours as the autofluorescence was very low. Instead the scans excitation: 675 nm and emission: 720 nm were used.

Results

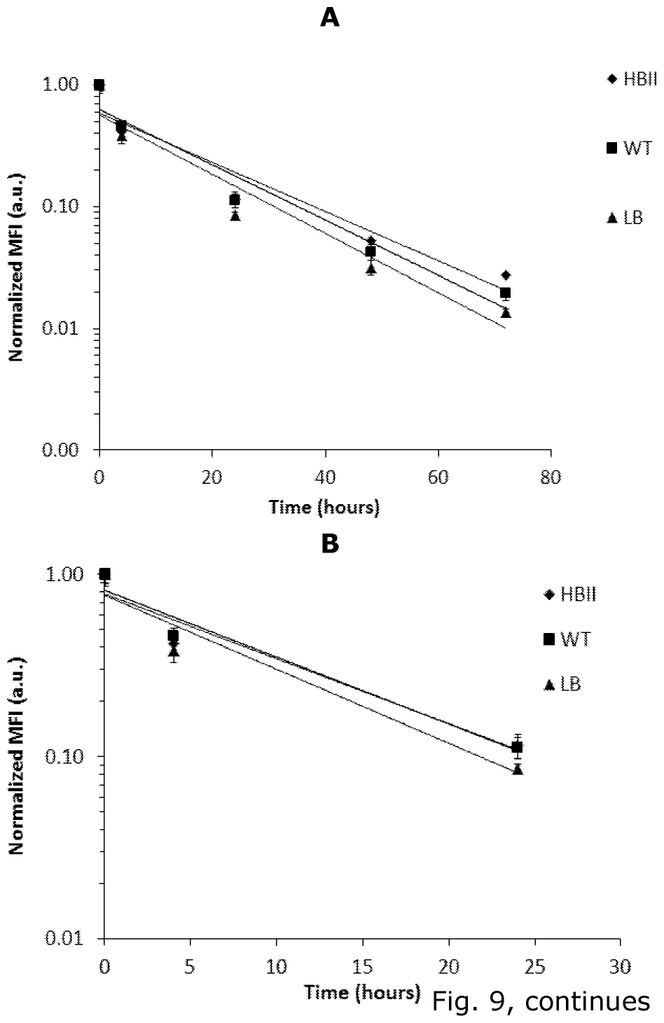

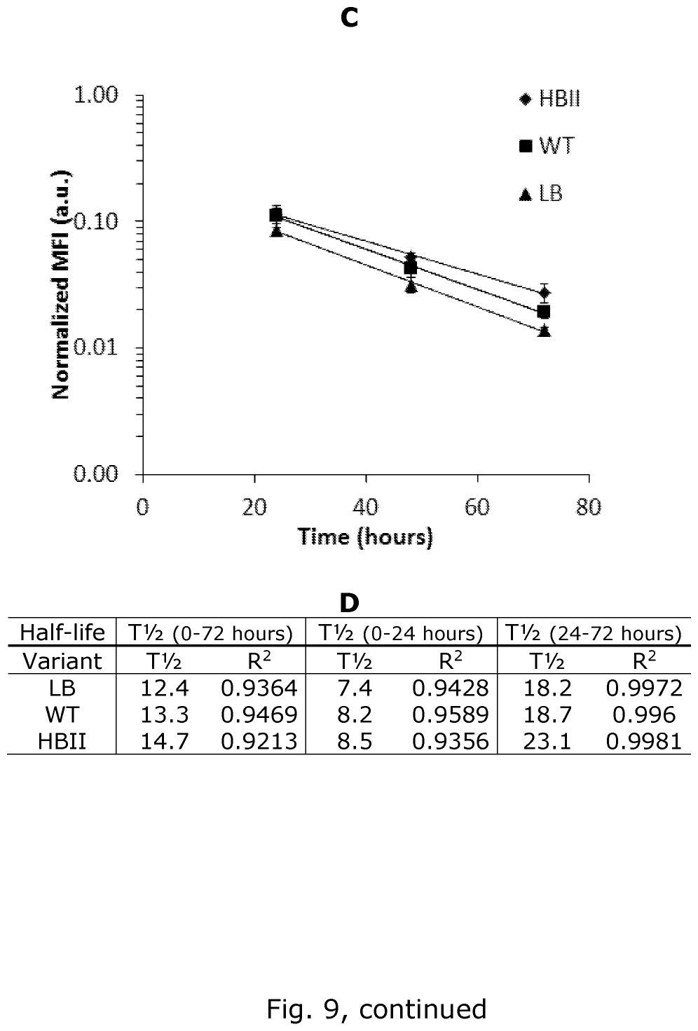

The results depicted in FIG. 9A shows the half-life of albumin variants and the corresponding exponential trend lines. It is observed that the trend line is steepest for the low-binder and levels out more for the high-binder, meaning a higher half-life for the high-binder.

The results in FIG. 9B show the initial phase of the half-life up to 24 hours. The high-binder and wild-type levels out more than the low-binder. The last phase of the half-life from 24-72 hours is depicted in FIG. 9C and shows that the high-binder II has a longer half-life compared to the wild-type and low-binder as the trend line is not as steep.

The table in FIG. 9D summarizes the calculated half-life values for all albumin variants and shows that a better fit is observed for the last phase trend lines (R² values ˜0.99) compared to both the entire time range (R²<0.95) and the initial phase (R²<0.96). The high-binder II shows the highest half-life of approximately 23 hours for the last phase. Therefore, these albumin variants behave as expected in vivo as the half-life is highest for the high-binder II and hence is useful for this study.

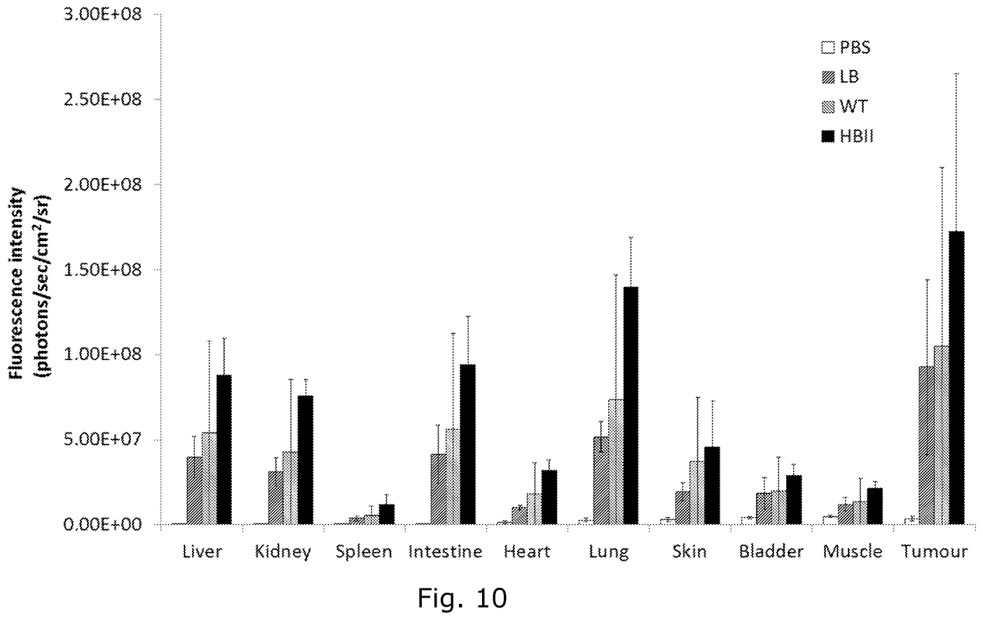

FIG. 10 shows the fluorescence intensity of the albumin variants in different organs and tumour and it is observed that all albumin variants accumulate in the tumour with the high-binder II to the highest degree.

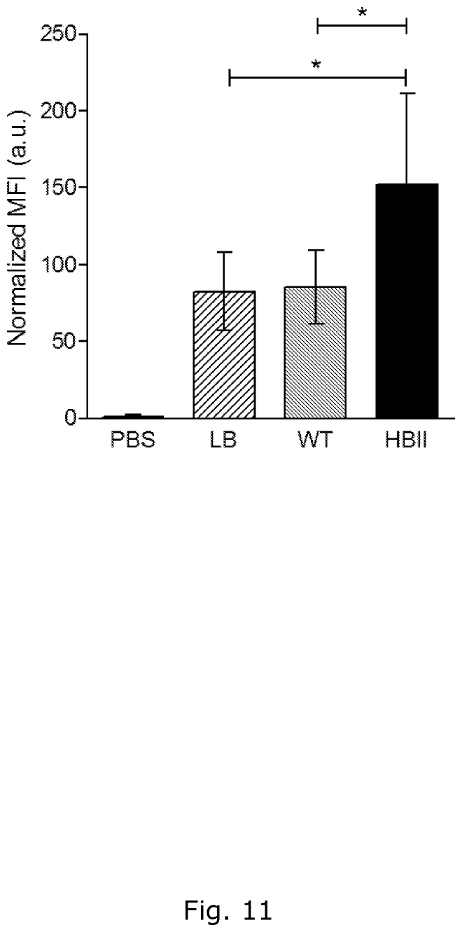

FIG. 11 shows the results of the fluorescence of the tumours alone and shows that the highest amount is observed with the high-binder II.

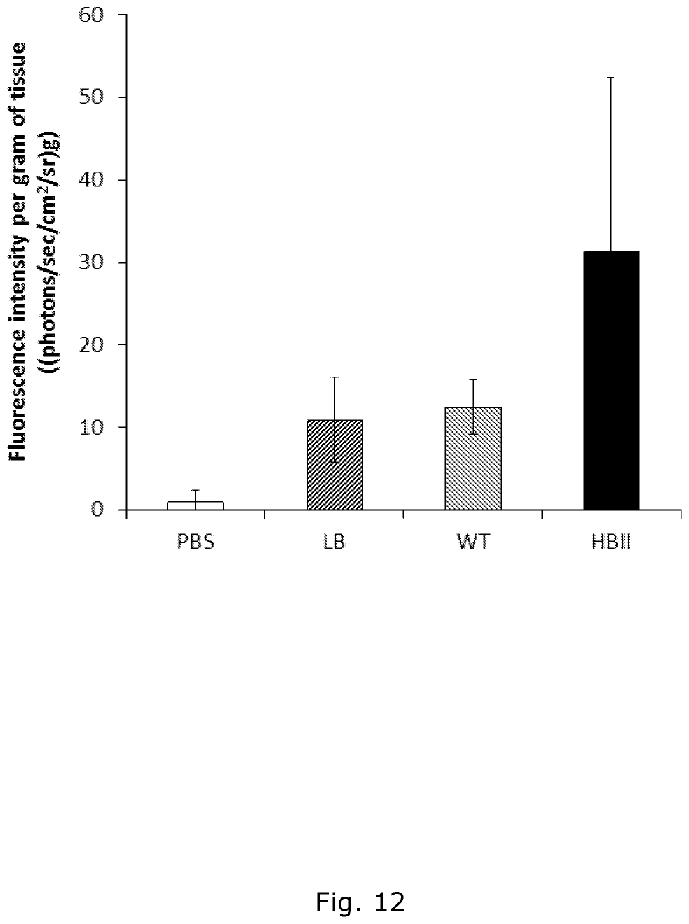

FIG. 12 shows the result when adjusting for the weight of the tumours and having a region of interest of constant size and shows the same pattern as in FIG. 11, namely of the highest accumulation present in the tumours by the high-binder II.

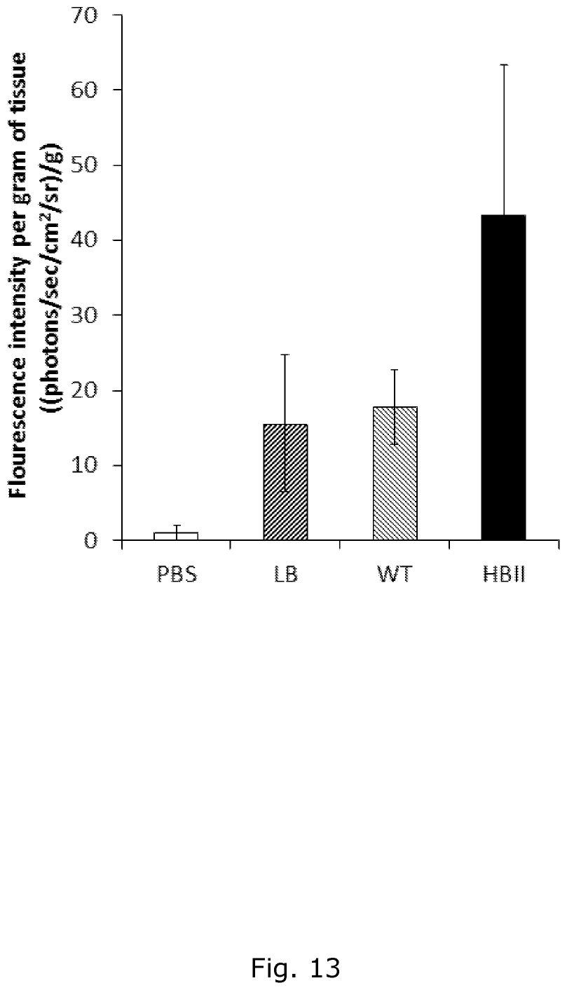

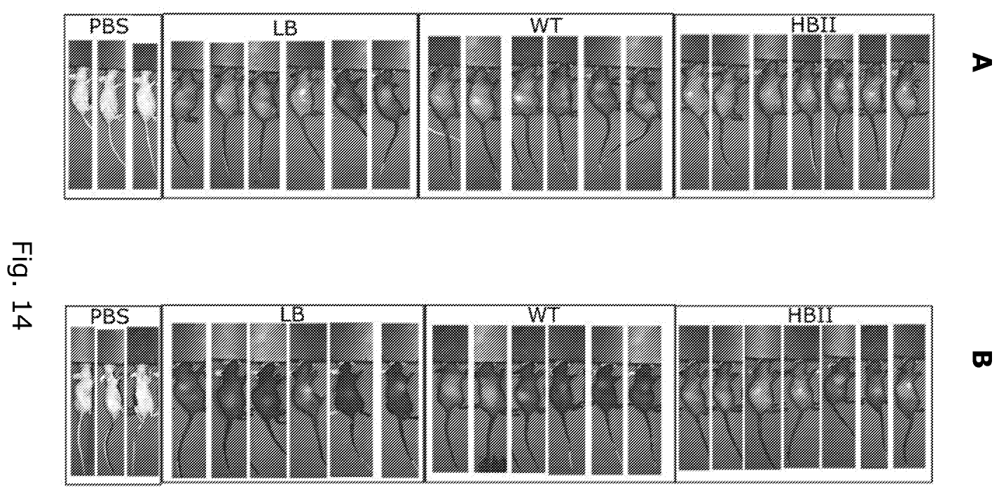

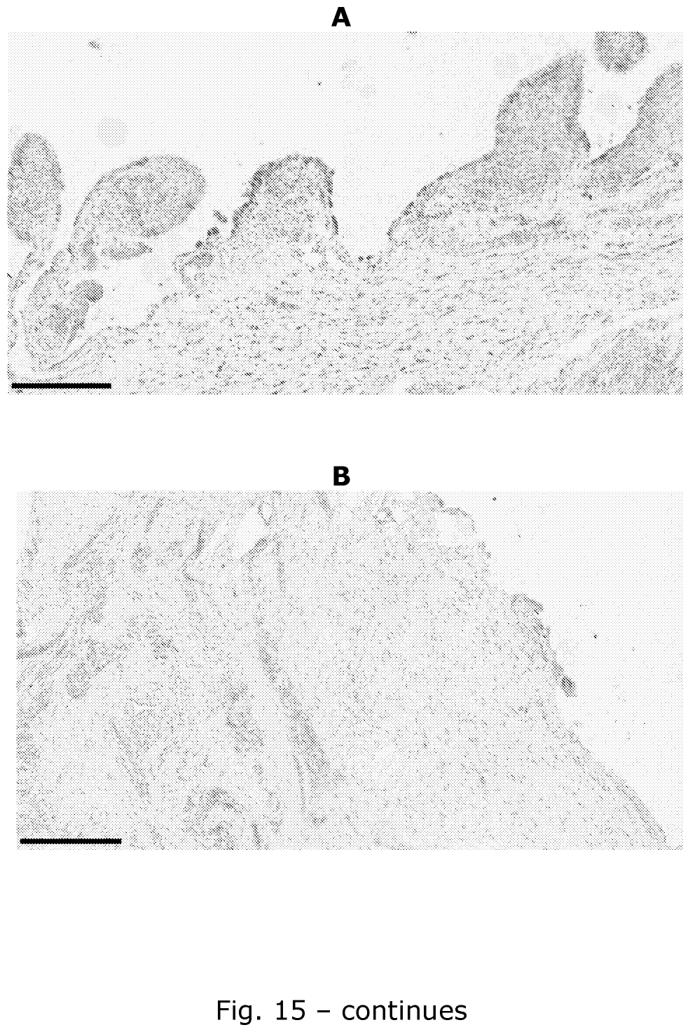

In FIG. 13 the entire tumour was chosen as region of interest but this did not affect the pattern remarkably. Still, the high-binder II accumulates to the same degree.

FIG. 14A gives a visual impression of the distribution of the fluorescent albumins in mice after 21 hours of administration, where they are all easily visualized.

FIG. 14B shows the albumin variant distribution after 72 hours and the fluorescence intensity is still observed around the tumour site for all albumin variants.

Conclusion

These results show indication of highest accumulation in tumours by the albumin FcRn high-binder variant suggestive of FcRn-driven accumulation.

### Example 5

Aim

Identification of FcRn expressing subtypes of inflammatory diseases.

Materials and Methods

Four rheumatoid arthritis samples were analysed.

The human rheumatoid arthritis tissue samples were provided by The Department of Biomedicine, Aarhus University, 8000 Aarhus C, Denmark.

Immunohistochemical Analysis of the Human Tissue Samples

Human tissue was formalin fixed and paraffin embedded (FFPE). FFPE human tissue sections were treated with Tissue Clear Xylene substitute (Tissue-Tek/Sakura Finetek) to de-paraffinise slides. Next, slides were rehydrated by gradually decreasing ethanol solutions from 100% to 75% and finally moved to running cold tap water.

Antigen retrieval was performed in citrate buffer pH 6.0 by heating in a microwave oven at 800 W for 8 min, followed by 560 W for 2×14 min and finally cooling for 20 min.

The staining procedure was performed on an Autostainer Link 48 Instrument (Dako). The slides were blocked using protein block (Dako) and stained with the primary polyclonal rabbit human FCGRT antibody (HPA012122, Sigma) in dilution 1:200.

For visualization the Dako EnVision™ FLEX (kit K8023) detection system was used. This included endogenous peroxidase blocking containing hydrogen peroxidase (EnVision™ FLEX Peroxidase-blocking Reagent, SM801), a polymer coupled with HRP and goat secondary antibody against rabbit immunoglobulins (EnVision™ FLEX/HRP, SM802), a DAB chromogen (EnVision™ FLEX DAB+Chromogen, DM827) and finally hematoxylin (EnVision™ FLEX Hematoxylin, K8008).

Results

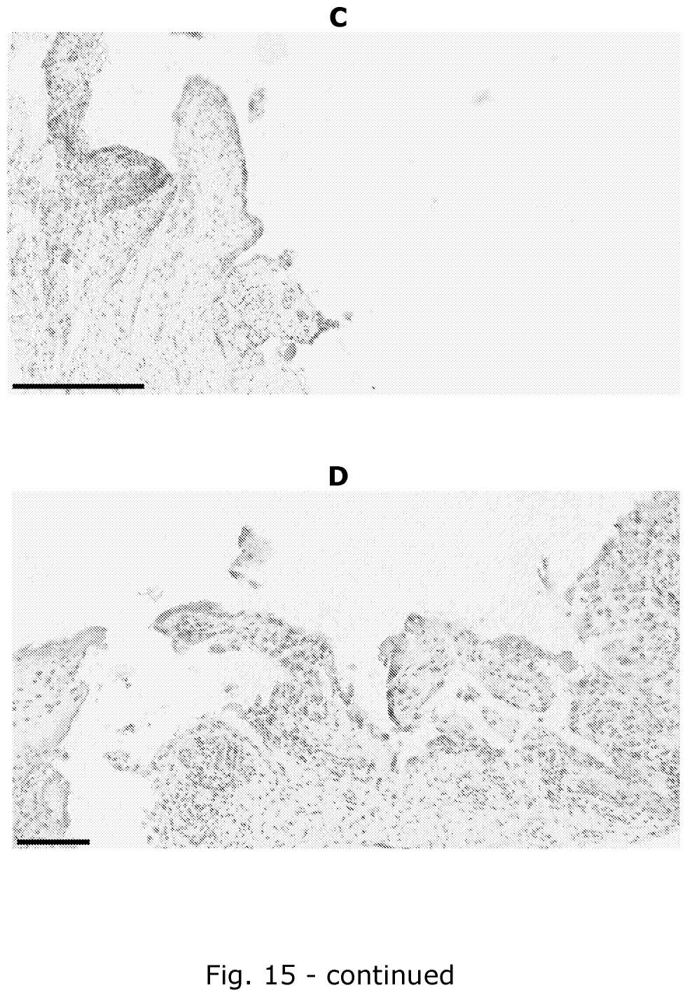

The results presented in FIG. 15 show representative examples of rheumatoid arthritis samples, which have been identified showing high levels of FcRn expression in the joints/synovial tissue.

Conclusion

It has been found that the FcRn receptor is expressed in the joints/synovial of the rheumatoid arthritis samples.

### Example 6

FcRn expression in patient cancer biopsies and bordering normal healthy tissue study.

Aim of Study

Detection of FcRn expression in more cancer types compared to normal healthy tissue.

The following cancer types were investigated:
Colorectal cancer (51)
Breast cancer (Luminal B (26) and Triple negative (51))
Kidney (Renal clear cell carcinoma (41))
Pancreatic cancer (47)
Cervical cancer (4)
Head and neck cancer (10)
Lung cancer (Non small cell lung cancer (11))
Ovarian cancer (33)
Bladder cancer (36)

Number of samples are shown in brackets.

Materials and Methods

Immunohistochemical Analysis of the Human Cancer Tissue Samples

Human tissue was formalin fixed and paraffin embedded (FFPE). FFPE human tissue sections were treated with Tissue Dear Xylene substitute (Tissue-25 Tek/Sakura Finetek) to de-paraffinise slides. Next, slides were rehydrated by gradually decreasing ethanol solutions from 100% to 75% and finally moved to running cold tap water.

Antigen retrieval was performed in citrate buffer pH 6.0 by heating in a microwave oven at 800 W for 8 min, followed by 560 W for 2×14 min and finally cooling for 20 min.

The staining procedure was performed on an Autostainer Link 48 Instrument (Dako). The slides were blocked using protein block (Dako) and stained with the primary polyclonal rabbit human FCGRT antibody (HPA012122, Sigma) in dilution 1:200.

For visualization, the Dako EnVision™ FLEX (kit K8023) detection system was used. This included endogenous peroxidase blocking containing hydrogen peroxidase (EnVision™ FLEX Peroxidase-blocking Reagent, SM801), a polymer coupled with Horseradish Peroxidase (HRP) and goat secondary antibody against rabbit immunoglobulins (EnVision™ FLEX/HRP, SM802), a DAB chromogen (EnVision™ FLEX DAB+Chromogen, DM827) and finally hematoxylin (EnVision™ FLEX Hematoxylin, K8008).

Scoring

The samples were grouped by a pathologist based on expression levels of FcRn and divided into four groups (Negative, Low, Moderate and High)

Results

The results presented in FIG. 16A-J show higher FcRn expression levels of FcRn compared to corresponding healthy tissue, for the following cancer types presented; colorectal cancer, breast cancer subtypes Luminal A and Triple Negative, Kidney cancer, Pancrea cancer, Cervix cancer, Head and neck cancer, Lung cancer (non small cell lung cancer), ovary cancer, and bladder cancer.

Conclusions

It is been found that in subtypes of colorectal cancer, breast cancer, Kidney cancer (Renal clear cell carcinoma), pancreatic cancer, bladder cancer, cervical cancer, Head and neck cancer, lung cancer (Non small cell lung cancer), and ovarian cancer over-expressing the FcRn receptor exist (compared to healthy/normal tissue).

It is believed that when such subtypes have been identified, the FcRn receptor could also be used as a binding agent for targeted cancer treatment of these specific subtypes.

Similar, the FcRn receptor may be used a binding moiety for the in vivo imaging of cancer, by coupling imaging agents to FcRn binding agents such as the albumins described in here.

Further, the FcRn receptor may be used a combined binding moiety for the in vivo imaging of cancer and for therapeutic applications, by coupling imaging agents and therapeutic agents to FcRn binding agents such as the albumins described in here.

### Example 7

Accumulation/targeting of engineered albumin high binders in human xenografts after intravenous injection in mice. Xenograft model 1: Bioluminescent xenografts.

Aim of Study

To investigate tumour accumulation of albumin variants.

Materials and Methods

Albumin Variants:

Alexa Fluor 680 labelled engineered albumin variants were provided by Albumedix Ltd. (Nottingham, UK).
HSA-K573P (High-binder I) (HBI) (SEQ ID NO: 4)
HSA-K500A (Low-binder) (LB) (SEQ ID NO: 6)

In Vivo Studies:

All animal experiments were performed at the Animal Facility at the Institute of

Biomedicine at Health, Aarhus University. All experiments were approved by the Danish Experimental Animal Inspectorate.

Cell Culture:

The human cancer cell line (MDA-MB-231/Luc), a luciferase-expressing breast cancer cell line, was used in this study. Cells were maintained in DMEM (4500 mg/L D-glucose, L-glutamine) (Gibco, Life Technologies, #41965-039) with 10% fetal bovine serum (FBS) (Gibco, Life Technologies, #10270-106), 0.1 mM MEM Non-essential amino acids (Gibco, Life Technologies, #11140-035), 2 mM L-glutamine (Lonza, #BE17-605E) and 1% penicillin and streptomycin (Gibco, Life Technologies, #15140-122).

Bioluminescent Tumour Xenografts

Breast cancer MDA-MB231/Luc cells (4×106 cells in 400 μl of 1:1 solution of PBS and matrigel, Geltrex™ LDEV-Free Reduced Growth Factor Basement Membrane Matrix, (Gibco, Life Technologies)) were injected subcutaneously into the right flank of each mouse (11 weeks old, female, Balb/canRj-Foxn1-nu, Janvier). Isoflurane was used as anesthesia for inoculation of tumour cells. Tumours were allowed to grow until visible (11 days) before they were randomly allocated into four group with approximately the same total tumour volume in each group (N=4 in treatment groups, N=3 in control group). Treatment was performed with 10 mg/kg fluorescent albumin for treatment groups and 150 μl PBS for the control group. Tumour sizes were measured on the days following the treatment by caliper measurements using the largest longitudinal (length) and width diameter of the tumour. The tumour volume was calculated by the formula (tumour volume=½ (length×width2).

At the end of the experimental time period, the mice were killed by cervical dislocation when anesthetized and the tumours and organs were collected. Blood and organs were analysed using the IVIS® Spectrum Bioimager (PerkinElmer, Waltham, Mass.). Background autofluorescence was eliminated using spectral unmixing and subsequent data analysis was carried out using Living Image software, version 4.3.1 (PerkinElmer).

Tumours, liver and kidney were snap-frozen for RNA isolation and fixed in neutral buffered formalin (10%) for preservation for immunohistochemistry studies. After 72 hours the tissue was dehydrated through a series of graded ethanol baths to displace water and next infiltrated with wax. The infiltrated tissue was then embedded into wax blocks.

Imaging and Quantification of Bioluminescence Data

The mice were injected subcutaneously with luciferin (D-luciferin, Caliper Life Sciences, Hopkinton, Mass.) at 300 mg/kg mouse body weight before being anesthetized with 3.5% isoflurane. Ten minutes after D-luciferin injection a whole body scan was performed with a Xenogen IVIS Spectrum imaging platform (PerkinElmer, MA), continuously applying 3.5% isoflurane anesthesia. A region of interest (ROI) was manually selected over relevant regions. The measured intensity was given as surface radiance (photons/s/cm2/sr) within a ROI.

Termination of the experiment was performed by cervical dislocation ten minutes after D-luciferin injection to ensure allowance of assessment of ex vivo bioluminescence. The tumours and organs of interest were collected and ex vivo imaged with the IVIS scanner within 40 minutes.

Albumin Injection and Sample Preparation

Alexa Fluor 680-labelled Albumin variants were injected intravenously at 2 mg/ml in the tail. After injection, blood samples were taken from the tongue (1 minute sample) and by tail nicking at 4 hours, 24 hours, 48 hours and 72 hours into heparin coated capillary tubes (Hirschmann® Laborgerate GmbH & Co.). The samples were transferred into microfuge tubes and centrifuged to fraction blood cells from serum. Samples were stored at 4° C. until scanning was performed.

Results

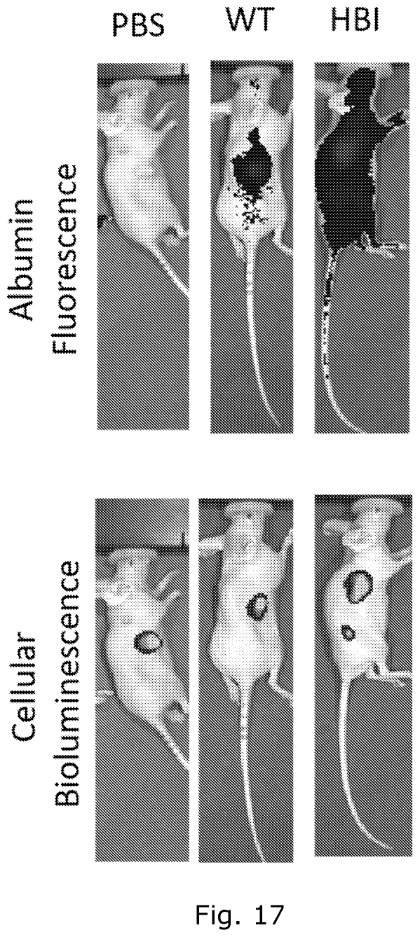

The top panel in FIG. 17 shows that the fluorescent albumin variant HBI exhibits greater accumulation than the WT variant at the tumour site (PBS control shows no fluorescence). The lower panel in FIG. 17 shows the cellular bioluminescence of MDAMB231/Luc cells after luciferin-D injection and depicts live tumour cells for each of the mice in the treatment groups PBS, WT and HBI.

The results in FIG. 17 show that the high-binding albumin variant is highly associated to the tumour site and that the fluorescence co-localizes with tumour cellular bioluminescence.

Conclusion

High targeting/accumulation in tumours is observed with the high-binder albumin variant. This clearly indicates that labelled FcRn binders (such as albumin high binders) can be used to visualize FcRn overexpression in vivo (such as cancer).

### Example 8

Flow Cytometric Cellular Association/Uptake of Fluorescent Albumin Variants to Colorectal Cell Line.

Aim of Study

Investigate association/uptake of fluorescent albumin variants in the colorectal cancerous HT-29 WT FcRn positive cell line and a HT-29 FcRn knockout (KO) cell line.

Materials and Methods

HT-29 WT and HT-29 knockout were seeded in well plates (24-well or 48-well) and allowed to reach confluency before experiment. Cells were treated with 8 μM fluorescent albumin labelled with Alexa488 in Hanks' balanced salt solution (HBSS) without phenol red using 1.0 M MES solution for 2 hours at 37° C., in a humidified atmosphere with 5% CO₂. Sample solution was removed and followed by a 3× wash using ice-cold HBSS. Cells were collected by trypsin treatment and centrifuged at 300 g for 5 min at 4° C., followed by another wash and resuspended in 400 μl of sterile-filtered PBS containing 1% bovine serum albumin (BSA) and 0.1% NaN₃. Samples were analyzed using a Gallios flow cytometer (Beckman Coulter) with a 488-nm laser and the 525/40 nm filter (FL1). Data processing was performed using Kaluza 1.2 software (Beckman Coulter).

Results

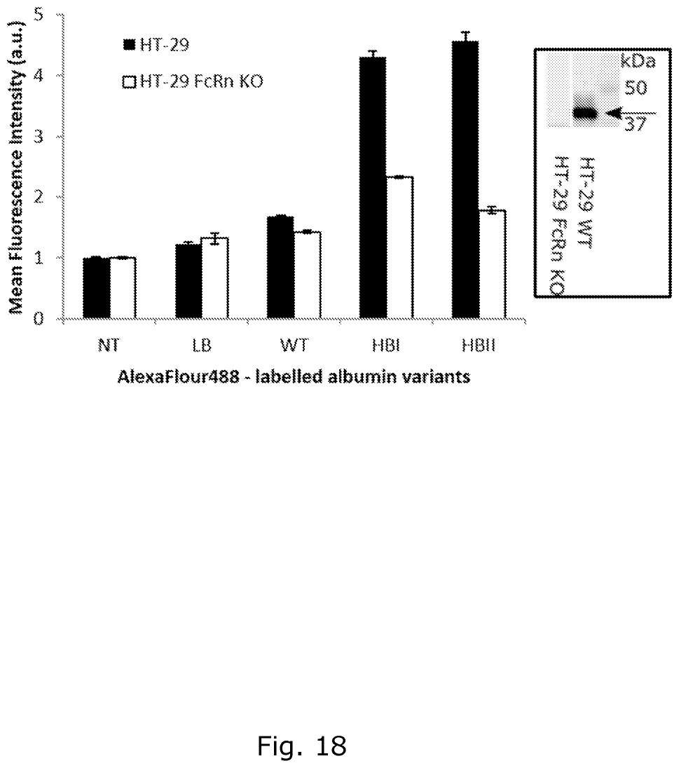

The results presented in FIG. 18, clearly shows that the FcRn high binding agents binds to (and/or taken into) the cell lines expressing FcRn, whereas the binding to (and/or uptake into) the FcRn knockout (KO) cell line is much lower. This demonstrates selective binding/uptake into FcRn-expressing cells with the high binding variant.

The insert shows a western blot that demonstrates no FcRn expression is detected in the HT-29 FcRn knockout (KO) cell line and that the HT-29 WT cell line is FcRn positive.

Conclusion

These results show that FcRn binders coupled to a drug can be used to selectively target cancers overexpressing FcRn. The coupled fluorescent molecules functions as a model for such drugs.

### Example 9

Western blot detection of FcRn expression in MDAMB231/Luc and HT-29 cell lines.

Aim of Study

Detection of FcRn expression in MDAMB231/Luc and HT-29 cell lines.

Materials and Methods

Western Blot

Harvesting of cells was performed using cold PBS containing HALT (Cat #78438, Thermo Fisher Scientific) and cells were scrabed off and centrifuged at 2500 rpm for 8 minutes. Homogenization was performed using pi3 kinase buffer with 0.1% SDS mixed with ceramic beads and shaking for 30 minutes at 4° C. and next 30 minutes shaking at room temperature with regular vortexing inbetween. Samples were centrifuged for 13300 rpm for 20 minutes and supernatant saved at 80° C. until further analysis. Protein concentration determination was performed using Micro BCATM Protein Assay kit (Cat #23235, Thermo Scientific). 10 ug of each sample was prepared with laemmli loading buffer and no heating. Western blots were performed using Criterion Stain-Free gradient gels using 4-15% (Bio-Rad, Hercules, Calif., USA) and visualized using ultraviolet (UV) exposure for 2 min using a Bio-Rad Chemidoc MP image apparatus. Protein transfer to 0.2 um PVDF membranes (Trans-Blot Turbo, Bio-Rad, Hercules, Calif., USA) was carried out using the Trans-Blot Turbo apparatus (Bio-Rad). A stain-free picture was taken for total protein quantification of the membrane and measured using ImageLab 4.1 software (Bio-Rad). Membranes were blocked with 1% BSA in TBS-T (0.01M Tris, 0.15 M NaCl, and 0.1% Tween 20) for 1 hour at room temperature. Primary antibodies were diluted in 1% BSA and 0.01% sodium azide and membrane incubated at 4° C. over night. Primary antibodies used were Anti-FCGRT used in 1:500 dilution (HPA012122, Sigma Aldrich). The membranes were washed three times in TBS-T and incubated with the secondary antibody goat-anti rabbit IgG-HRP (sc-2054, Santa Cruz Biotechnology) diluted 1:10000 for 1.5 hours at room temperature. The membrane was washed three times in TBS-T and incubated with Clarity Western ECL substrate (Bio-Rad) and the bands visualized using the ChemiDoc MP image apparatus and analysed using ImageLab 4.1. Appropriate loading control was based on total protein analysis from membranes (data not shown).

Results

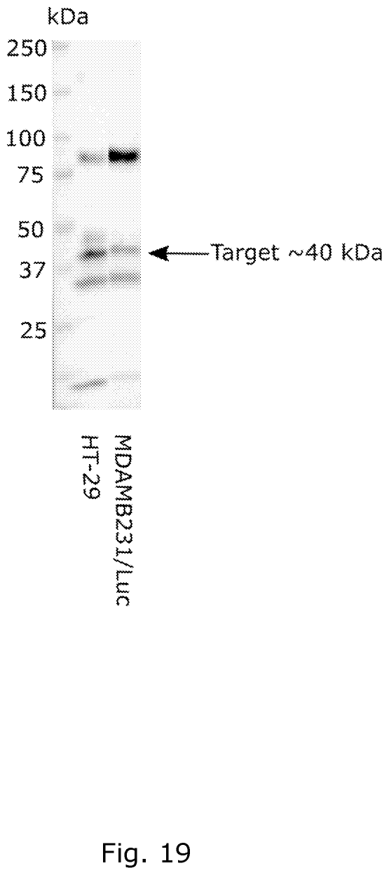

The results presented in FIG. 19 show that the MDAMB231/Luc and HT-29 cell lines express FcRn.

Conclusion

The MDAMB231/Luc and HT-29 cell lines are useful cell lines for creating mice xenograft and performing in vivo FcRn binding agent experiments or cancer treatment.

1. A method of subtyping a cancer, staging a cancer, or predicting the risk of developing a cancer, the method comprising:
providing a biological sample from a subject;
measuring the level of FcRn in said sample; and
comparing said measured level to a reference level;
wherein a higher level of FcRn in said sample compared to the reference level is indicative of an FcRn up-regulated subtype/stage; and wherein a level equal to or lower than said reference level is indicative of an FcRn normal subtype/stage or FcRn down-regulated subtype/stage;
or
wherein a higher level of FcRn in said sample compared to the reference level is indicative of an increased risk of developing a cancer; and wherein a level equal to or lower than said reference level is not indicative of an increased risk of developing a cancer.

2-34. (canceled)

35. The method of claim 1, wherein a higher level of FcRn in said sample compared to the reference level is indicative of an increased risk of developing a cancer; and wherein a level equal to or lower than said reference level is not indicative of an increased risk of developing a cancer.

36. The method according to claim 1, wherein said method is subtyping/identifying a cancer susceptible to treatment by an FcRn binding agent.

37. The method according to claim 1, wherein said cancer type is selected from the group consisting of breast cancer, colorectal cancer, lung cancers, pancreatic cancers, liver cancers, intestinal cancers, prostate cancers, bladder cancers, kidney cancers, such as renal clear cell carcinoma, ovarian cancers, cervical cancers, adenocarcinomas, squamous cell carcinomas, head and neck cancer and ear, nose, and throat cancers.

38. The method according to claim 1, wherein said biological sample is selected from the group consisting of tissue biopsies, blood, stool (faeces), urine, pleural fluid, saliva, gall, bronchial fluid, oral washings, ascites, pus, cerebrospinal fluid, follicular fluid, tissue and mucus.

39. The method according to claim 1, wherein the biological sample is a cancer sample.

40. The method according to claim 1, wherein said reference level is the level of FcRn of a normal sample (non-cancer) of the same type or an average level from several normal samples.

41. The method according to claim 1, wherein said reference level is the level of FcRn of a normal sample (non-cancer), and wherein said normal sample is from tissue bordering said biological sample, or tissue distant from said biological sample.

42. The method according to claim 1, wherein said level is measured using an FcRn binding agent.

43. The method according to claim 1, wherein said level is measured using an FcRn binding agent and wherein said FcRn binding agent is selected from the group consisting of WT albumins, albumin variants, FcRn antibodies, IgG's, peptides, proteins, and nucleic acids.

44. The method according to claim 1, wherein said level is measured using an FcRn binding agent and wherein said FcRn binding agent is an albumin variant.

45. The method according to claim 1, wherein said level is measured using an FcRn binding agent, and wherein the binding agent comprises a detectable label.

46. The method according to claim 1, wherein said level is measured using an FcRn binding agent and wherein said FcRn binding agent is an albumin variant having a higher binding affinity to FcRn than the WT version of albumin (SEQ ID NO: 1).

47. The method according to claim 1, wherein said level is measured using an FcRn binding agent, and wherein the binding agent comprises a detectable label and wherein the detectable label is chosen from radioisotopes, enzymes having detectable products, fluorophores, chemiluminescent compounds, magnetic particles, microparticles, microspheres, nanoparticles, nanospheres, biotin, streptavidin, or digoxin.

48. A method for treating a subject having a cancer over-expressing FcRn, the method comprising administering to the subject an FcRn binding agent coupled to a therapeutic agent.

49. The method according to claim 48, wherein said FcRn binding agent is an albumin variant.

50. The method according to claim 48, wherein the therapeutic agent is a radionuclide, an anti-cancer drug, Actinomycin-D, Aldesleukin, Alemtuzumab, alkane sulfonates, Alkeran, Amsacrine, Anastrozole, Anastrozole, anthracyclines, antimetabolites, Ara-C, Arsenic trioxide, Asparaginase, Azathioprine, BCG, Bicalutamide, BiCNU, Bleomycin, Bortezomib, Busulfan, Busulphan, Capecitabine, Carboplatin, Carboplatinum, Carmustine, CCNU, Cetuximab, Chlorambucil, Chloramphenicol, chorionic, Ciclosporin, Cidofovir, Cisplatin, Cladribine, Coal tar containing products, Colchicine, CPT-11, Cyclophosphamide, Cytarabine, Cytosine arabinoside, Cytoxan, Dacarbazine, Dactinomycin, Danazol, Dasatinib, Daunorubicin, Dexrazoxane, Diethylstilbestrol, Dinoprostone, Dithranol containing products, Docetaxel, Doxorubicin, DTIC, Dutasteride, Epirubicin, Estradiol, Estramustine, Ethyleneimine, Etoposide, Exemestane, Finasteride, Floxuridine, Fludarabine, Fluorouracil, Flutamide, folate analogs, Fotemustine, Ganciclovir, Gemcitabine, Gemtuzumab, Gonadotrophin, Goserelin, Herceptin, Hexamethylamine, hormonal agents, Hydroxycarbamide, Hydroxyurea, Idarubicin, Ifosfamide, Imatinib mesylate, Interferon containing products (including peginterferon), Irinotecan, Leflunomide, Letrozole, Leuprorelin acetate, Lomustine, Mechlorethamine, Medroxyprogesterone, Megestrol, Melphalan, Menotropins, Mercaptopurine, Methotrexate, Mifepristone, Mitomycin, Mitotane, Mitoxantrone, Methotrexate (MTX), Mycophenolate mofetil, Nafarelin, nitrogen mustards, nitrosorueas, Oestrogen containing products, Oxaliplatin, Oxytocin, Paclitaxel, Pamidronate, Pentamidine, Pentostatin, platinum compounds, Plicamycin, Podophyllyn, Procarbazine, Progesterone containing products, purine analogs, pyrimidine analogs, Raloxifene, Raltitrexed, Ribavarin, Rituximab, Sirolimus, Steroids, STI-571, Streptozocin, syntocinon, syntometrine, Tacrolimus, Tamoxifen, taxanes, Temozolomide, Teniposide, Testosterone, Tetrazine, Thalidomide, Thioguanine, Thiotepa, Tomudex, topoisomerase inhibitors, Topotecan, Toremifene, Trastuzumab, Treosulphan, Trifluridine, Trimetrexate, Triptorelin, Valganciclovir, Vidaradine, Vinblastine, vinca alkaloids, Vincristine, Vindesine, Vinorelbine, VP-16, Xeloda, or Zidovudine.

51. A method for in vivo imaging a subject having a cancer over-expressing FcRn, the method comprising:
a) administering to the subject an FcRn binding agent coupled to a detectable moiety, and
b) imaging the subject to detect said FcRn binding agent coupled to a detectable moiety bound to the cancer over-expressing FcRn.

52. The method according to claim 51, wherein the detectable moiety is a radioactive detectable moiety suitable for imaging using PET or SPECT, or a non-radioactive detectable moiety suitable for imaging.

53. The method according to claim 51, wherein the detectable moiety is a radionuclide selected from the group consisting of ¹¹C, ¹⁵O, ¹⁸F labelled fludeoxyglucose, ⁶⁴Cu, ⁶⁸Ga, ⁶⁶Ga, ⁶⁰Cu, ⁶¹Cu, ⁶²Cu, ⁸⁹Zr, ¹²⁴I, ⁷⁶Br, ⁸⁶Y, ^{94m}Tc, ¹³¹I, ^{G67}Ga, ¹¹¹In, ¹²³I, and ^{99m}Tc.

54. The method according to claim 1, further comprising administering to the subject an FcRn binding agent coupled to a therapeutic agent when a higher level of FcRn in said sample compared to the reference level is measured.


SEQUENCE LISTING (7 sequences)

SEQ ID NO: 1 - protein, 585 aa, Homo sapiens [sequence omitted]

SEQ ID NO: 2 - protein, 290 aa, Homo sapiens
  misc_feature (1)..(290): Truncated heavy chain of the major histocompatibility complex class I-like Fc receptor (FCGRT) (together, SEQ ID No. 2 and SEQ ID No. 3 form FcRn)
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVEL

SEQ ID NO: 3 - protein, 119 aa, Homo sapiens
  misc_feature (1)..(119): Beta-2-microglobulin (together, SEQ ID No. 2 and SEQ ID No. 3 form FcRn)
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 4 - protein, 585 aa, Artificial Sequence
  HSA K573P
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 5 - protein, 585 aa, Artificial Sequence
  HSA E492G+K573P+K574H+Q580K
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 6 - protein, 585 aa, Artificial Sequence
  HSA K500A
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPAEFNAETFTFHADICTLSEKERQIKKQTALVELVKHKPKATKEQLKAVMDDFAAFVEKCCKADDKETCFAEEGKKLVAASQAALGL

SEQ ID NO: 7 - protein, 585 aa, Artificial Sequence
  HSA-K500A+ H464Q
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLQEKTPVSDRVTKCCTESLVNRRPCFSALEVDETYVPAEFNAETFTFHADICTLSEKERQIKKQTALVELVKHKPKATKEQLKAVMDDFAAFVEKCCKADDKETCFAEEGKKLVAASQAALGL